(19) United States
(12) Patent Application Publication
(10) Pub. No.: US 2003/0113390 A1
(43) Pub. Date: Jun. 19, 2003

(54) Composition comprising soy protein, dietary fibers and a phytoestrogen compound and use thereof in the prevention and/or treatment of various diseases

(76) Inventor: Lars Henrik Hoie, London (GB)

Correspondence Address: BROWDY AND NEIMARK, P.L.L.C., 624 Ninth Street, N.W., Washington, DC, 20001, US

(21) Appl. No.: 10/254,636

(22) Filed: Sep. 26, 2002

Related U.S. Application Data
(63) Continuation of application No. 09/502,598, filed on Feb. 11, 2000.
Application No. 09/502,598 is a continuation-in-part of application No. PCT/DK99/00655, filed on Nov. 25, 1999.
(63) Continuation of application No. PCT/IB99/01992, filed on Nov. 25, 1999.
(63) Continuation of application No. PCT/IB99/01997, filed on Nov. 25, 1999.
(63) Continuation of application No. PCT/IB99/01998, filed on Nov. 25, 1999.
(60) Provisional application No. 60/110,505, filed on Dec. 1, 1998.
(60) Provisional application No. 60/110,506, filed on Dec. 1, 1998.

(30) Foreign Application Priority Data
Nov. 25, 1998 (DK) PA 1998 01555
Jun. 16, 1999 (DK) PA 199900855
Nov. 25, 1998 (DK) PA 1998 01556
Jun. 16, 1999 (DK) PA 1999 00856
Nov. 25, 1998 (DK) PA 1998 01557

(51) Int. Cl. A61K 35/78

(52) U.S. Cl. 424/757

(57) ABSTRACT

A composition comprising (a) soy protein, (b) a phytoestrogen compound, and (c) dietary fibres is provided. The soy protein (a) is present in an amount of at least 45 weight percent of the total protein content of the composition, said total protein content providing at least 15 percent of the total energy content of the composition. The phytoestrogen compound (b) is preferably a naturally occurring isoflavone and is present in an amount of more than 0.10 weight percent of the soy protein, and the dietary fibres (c) are preferably soybean fibres and are present in an amount of more than 6 weight percent of the total weight of the nutritional composition on a dry basis. The composition is useful for treating various diseases. Alternatively, the phytoestrogen is more than 0.55 weight percent of the soy protein and the dietary fibers are more than 4 weight percent of the total weight.

The present application is a continuation of PCT/IB99/01992, PCT/IB99/01997 and PCT/IB99/01998, all of which are hereby incorporated by reference.

### FIELD OF THE INVENTION

The present invention relates to soy protein, phytoestrogens and dietary fibers and compositions thereof suitable for preventing, treating and/or alleviating cardiovascular diseases such as hypercholesterolemia, hypertriglyceridemia, hyperlipidemia, arteriosclerosis, hypertension and related cardiovascular diseases, for preventing and/or treating type 2 diabetes and/or the metabolic syndrome, and for preventing, treating and/or alleviating pulmonary diseases. The present invention also pertains to the use of such compositions in the prevention and/or treatment of a cardiovascular disease in a subject suffering from type 2 diabetes

A composition according to the present invention is particularly useful in preventing and/or reducing the influx of triglycerides and/or cholesterol into the arterial wall and/or reducing the accumulation of cholesterol in the arterial wall of subjects at high risk for developing cardiovascular disease or subjects already suffering from a cardiovascular disease such as atherosclerosis or diabetic subjects. A composition according to the present invention is also useful for lowering serum levels of total cholesterol and/or LDL-cholesterol and/or triglycerides and/or homocystein and/or for increasing serum levels of HDL-cholesterol and/or for improving the serum HDL/LDL-ratio in subjects at risk for developing cardiovascular diseases and in subjects already suffering from an arteriosclerotic condition such as e.g. atherosclerosis or a related cardiovascular disease. A composition according to the present invention is also useful in lowering serum levels of glucose and/or total cholesterol and/or LDL-cholesterol and/or triglycerides in diabetic subjects. A composition according to the present invention is also useful in treating e.g. chronic obstructive pulmonary disease (COPD), inflammation of the airways, asthma, bronchoconstriction, bronchitis, and small airways disease.

In addition the present invention relates to the use of these compositions as a medicament and/or in the manufacture of a medicament for treating a subject suffering from cardiovascular diseases, more particularly hypercholesterolemia, hypertriglyceridemia, hyperlipidemia, arteriosclerosis, hypertension and/or related cardiovascular diseases. The present invention also relates to the use of these compositions as a medicament and/or in the manufacture of a medicament for treating type 2 diabetes and/or the metabolic syndrome and/or a cardiovascular disease in a subject suffering from type 2 diabetes. Furthermore, the present invention also relates to the use of these compositions as a medicament and/or in the manufacture of a medicament for treating a subject suffering from a pulmonary disease, more particularly chronic obstructive pulmonary disease (COPD), inflammation of the airways, asthma, bronchoconstriction, bronchitis, and/or small airways disease.

The present invention also concerns use of a composition according to the present invention in the prevention and/or treatment of said diseases and disorders and for lowering serum levels of total cholesterol and/or LDL-cholesterol and/or triglycerides and/or homocystein in subjects. In addition, the present invention also provides methods for preventing, treating, prophylactically treating and/or alleviating by therapy said diseases and disorders.

### BACKGROUND OF THE INVENTION

Lipid metabolism involves biosynthesis and degradation of e.g. fatty acids, triglycerides and cholesterol. Ingested triglycerides are hydrolyzed in the small intestine and hydrolysis products are absorbed by the intestinal mucosa. Due to the relative insolubility of dietary lipids in water, lipid digestion and absorption is facilitated by the action of detergent substances such as bile acids secreted from the gallbladder. Bile acids are essential for lipid digestion and absorption through the intestinal mucosa.

Triglycerides and cholesterol synthesized in the liver are transported in the bloodstream to peripheral tissues by transport proteins called lipoproteins. Lipoproteins are tiny vesicles coated by apoproteins, phospholipids and free cholesterol and with an interior consisting of the more hydrophobic lipids, cholesteryl esters and triglycerides. Apoproteins and lipoproteins are primarily synthesized in the liver. The lipoproteins are capable of performing an apoprotein mediated binding to a receptor on the surface of a cell into which the entire lipoprotein particle is taken up and further metabolized.

Several different families of lipoproteins have been characterized and are traditionally classified by their density as determined by centrifugation. A standard lipoprotein classification scheme may include in increasing order of density, very low-density lipoprotein (VLDL), intermediate-density lipoprotein (IDL), low-density lipoprotein (LDL), and high-density lipoprotein (HDL).

VLDL contains approximately 60 to 65 percent triglycerides and 5 to 10 percent cholesterol, lecithin and protein. They are relatively large and function in the transport of triglycerides from the liver to tissue. LDL contains approximately 40 to 50 percent cholesterol and 10 to 15 percent triglycerides, lecithin and protein. They are somewhat smaller than VLDL and also function in the transport of cholesterol from the liver to tissue. HDL contains roughly 75 percent lecithin and protein, while the rest is made up of cholesterol and a small amount of triglycerides. They function in the transport of cholesterol from tissue to the liver and, as such, have the opposite function of LDL. Cholesterol esters cannot readily traverse cellular membranes and are taken up by cells in a receptor-mediated process. Once bound to the LDL receptor, the LDL-particle is internalized by means of endocytosis, and cholesterol and fatty acids are released and further metabolized.

Ongoing investigations of the LDL receptor mediated internalization of cholesterol have generated a better understanding of the relationship between dietary cholesterol, plasma cholesterol levels, and the condition of artherosclerosis. It is believed that the white blood cells that accumulate cholesterol at sites of arterial injury contain a receptor termed a scavenger receptor. Like the LDL receptor, this scavenger receptor acts by the mechanism of endocytosis and mediates internalization of various extracellular materials. However, the scavenger receptor is indiscriminatory and takes up many different types of extracellular materials including oxidized LDL particles containing cholesterol. In contrast to the LDL receptor, the scavenger receptor is not down-regulated by a high concentration of cholesterol in the cell.

In addition to the above-mentioned lipoproteins, the organism also contains a type of lipoproteins called chylomicrons. Chylomicrons contain 90 to 95 percent triglycerides and only a small amount of cholesterol, lecithin and protein, and they function in the transport of triglycerides from the small intestine to e.g. muscles, liver and heart.

The metabolism of cholesterol in the human organism is closely linked to the synthesis, transport and degradation of triglycerides. Cholesterol is an essential lipid component in all mammalian cells. It is used to regulate the fluidity of cellular membranes and serves as a precursor for certain hormones, vitamin D and bile acids. Cholesterol is synthesized in the liver and is transported with the blood to peripheral tissues by lipoproteins. The liver has a dual function in the metabolism of cholesterol since it is capable of both synthesizing cholesterol and converting surplus cholesterol into bile acids. It is also capable of excreting cholesterol into the bile.

Bile acids have ampholytic characteristics and contain both hydrophobic and hydrophilic surfaces. This ampholytic character facilitates a bile acid mediated emulsification of lipids into micelles. The formation of micelles allows digestive attacks by water-soluble enzymes and facilitates lipid absorption through the mucosal cells of the intestine. Bile acids are secreted from the liver and stored in the gallbladder before being passed through the bile duct and into the intestine. Biosynthesis of bile acids represents a major metabolic fate of cholesterol and accounts for more than half of the approximately 800 mg cholesterol that is normally metabolized per day in a normal adult. Even though bile acids in an amount of 400 mg are synthesized each day, significantly more than this amount is secreted into the intestine. Most of the bile acids that are secreted into the upper small intestine are absorbed in the lower small intestine and are recycled to the liver. The process of enterohepatic circulation may amount to as much as 20 to 30 g of bile acids per day. In contrast, daily elimination of bile acids in the feces amounts to just 0.5 g or less.

Cholesterol acts on three different levels of regulation of its own synthesis. Firstly, it suppresses endogenous cholesterol synthesis by inhibiting HMG-CoA reductase. Secondly, it activates acyl-CoA:cholesterol acyltransferase (ACAT) which is involved in the synthesis of cholesterol esters from cholesterol and fatty acids bound to acyl-CoA. Thirdly, cholesterol regulates synthesis of the LDL receptor. Accordingly, a decreased synthesis of LDL receptors will ensure that a cell in which a sufficient amount of cholesterol is already present does not take up cholesterol. This may explain why excessive dietary cholesterol generates a rapid elevation of cholesterol levels in the blood.

The presence of increased amounts of cholesterol in the blood is known to be positively correlated to arteriosclerosis, a condition commonly attributed to the deposition on the inner lining of ari arterial wall of plaque in the form of cholesterol and fats. Arteriosclerosis is a common term for a group of conditions related to the arterial system and leading to an increased arterial wall thickness and a subsequent loss of elasticity. Three main groups of arteriosclerosis frequently referred to are atherosclerosis, Mönckeberg's mediasclerosis and arteriolosclerosis. Atherosclerosis is most frequently observed in the aorta and in the main arteries connected thereto, in the coronary arteries and in the arteries of the brain. Mönckeberg's mediasclerosis leads to a narrowing of the media of the arteries of the extremities, and arteriolosclerosis is related to a narrowing of the small arteries and arterioles caused mainly by hypertension. Other arteriosclerotic manifestations include hyperlipidemia, hypercholesterolemia, hypertriglyceridemia, hyperglycemia, hypertension, and hyperinsulinemia

One commonly occurring arterial condition is that of atherosclerotic cardiovascular disease. The condition may eventually progress through several stages. A normal structure of an artery is characterized by discrete focal numbers of adhering monocytes, some intimal foam cells, and some intimal smooth muscle cells, or intimal cell masses at bifurcations. A fatty streak may occur non-symptomatically and involve a layer of foam cells. As arteriosclerosis progresses, the cells making up the inner wall of an artery will gradually start to harden due to the deposition of lipid and calcium and proliferation of smooth muscle cells, and the cells may eventually become degenerated. As the wall of an artery thickens, hardens, and lose its elasticity during arteriosclerosis, the blood vessels may develop twists and turns and become narrowed so that the heart must work harder to pump the usual amount of blood through the arteries. The condition may regress or it may evolve into a formation of e.g. fibrous plaque. Fibrous plaque is a slowly reversible condition that may develop further into a complicated lesion.

The cellular degeneration is likely to result in a fracture of the arterial wall which in turn leads to the formation of a deposit of calcium, platelet formation and a gradual development of scar tissue further contributing to both cellular degeneration and a substantially reduced elasticity of the arterial wall. Atherosclerosis characterized by a restricted flow of blood through a coronary artery may lead to the development of coronary heart disease. A complicated lesion of an artery is often symptomatic, hardly reversible and may, in severe cases such as thrombosis, be lethal. A decreased flow of blood through an artery may lead to the formation of blood clots and this may eventually lead to thrombosis. If a blood clot forms in a coronary artery, the interruption of the blood flow may result in the death of part of the heart muscle and cause the extremely painful chest pains associated with a heart attack.

Arteriosclerotic symptoms largely depend on the arteries and tissue affected. When arteriosclerosis occurs in the arteries leading to the brain, the decrease in blood flow and oxygen can cause mental confusion and personality changes. A stroke may occur, if an artery in the brain that has been weakened by a rupture or a blood clot prevents blood from flowing to the brain. This may possibly result in e.g. partial paralysis, loss of speech, and sometimes even death. A decrease in the flow of blood through the coronary arteries results in a shortage of oxygen to the heart muscle and causes chest pains and a painful condition called angina pectoris. Angina pectoris is usually caused by a narrowing or an obstruction of a coronary artery. An attack of angina pectoris may be caused by stress or result from physical activities that require an increased supply of blood to the heart.

Although it is well established that cholesterol, lipids and lipoproteins all contribute to the progression of various arteriosclerotic conditions in diabetic and non-diabetic subjects alike, little is known about the causes of arteriosclerosis. Hereditary conditions clearly play a role in some cases and several socio-economical and life style related factors such as smoking, hypertension, dietary habits and continual stress also contribute to the development of arteriosclerosis.

There is at present no simple cure or medical treatment for arteriosclerosis and doctors usually advise patients to follow a low fat diet, to stop smoking and to exercise regularly. Patients suffering from hypercholesterolemia may be classified into four risk groups: (i) manifest coronary artery disease, (ii) other forms of atherosclerotic vascular disease, (iii) other risk factors for coronary artery disease in the absence of established atherosclerotic cardiovascular disease, and (iv) isolated hypercholesterolemia in the absence of other risk factors. The recommended treatment regimen for risk group (iv) is to give general advice together with the laboratory results to those patients having a total cholesterol level of 5.0-6.4 mmol/l, without any further follow-up. To patients with cholesterol levels in the range of 6.5-7.9 mmol/l and LDL levels >5.0 mmol/l or an LDL/HDL ratio >5.0, only non-pharmacological treatment is offered.

Drug treatment of cardiovascular diseases may include the use of calcium channel blockers to expand the arteries so that blood can flow more freely, and anticoagulants to prevent blood clots from forming in diseased arteries. Some studies indicate that compounds such as acetylsalicylic acid and sulphinpyrazone, which may reduce and/or inhibit clotting by reducing platelet reactivity, may also prevent formation of a thrombus. In advanced cases, surgery to replace diseased blood vessels with grafts of healthy arteries may be necessary.

Also, various lipid-lowering drugs have been advocated, as some studies have shown or indicated that even for otherwise healthy patients suffering from mild or moderate hypercholesterolemia, coronary morbidity and mortality is reduced when they are treated with such lipid-lowering drugs. The most widely used lipid-lowering drugs in recent years have been statins, such as HMG-CoA-reductase-inhibitors, bile acid resins, fibrates, nicotinic acid derivatives and various fish oil concentrates with a high content of ω-3-fatty acids.

Many cardiovascular risk factors are abnormally high in individuals suffering from type 2 diabetes and these individuals are consequently at an increased risk of developing various arteriosclerotic vascular diseases. At the time of diagnosis of type 2 diabetes, the existence of arteriosclerotic manifestations is already pronounced in many individuals and may include hypercholesterolemia, hypertriglyceridemia, hyperlipidemia, hyperglycemia, hypertension, and hyperinsulinemia.

In type 2 diabetes, an impaired insulin secretion as well as decreased insulin sensitivity is present. As a result of this, glucose is present in excessive amounts in the bloodstream in association with either low, normal or even high insulin levels.

Serum levels of glucose vary quite significantly depending on the nutritional status of a subject. Following a dietary intake rich in glucose containing carbohydrates, several homeostatic mechanisms are capable of promoting glucose uptake into cells as well as facilitating the metabolism of glucose leading e.g. to the synthesis of glycogen in the liver and muscles. When glucose levels subsequently fall some time after a meal, other regulatory mechanisms initiates the release of glucose from glycogen and initiates gluconeogenesis in order to maintain the blood glucose levels within the required limits.

Some homeostatic mechanisms are dependent on the action of hormones, and the most important hormone promoting glucose uptake and metabolism is insulin. In contrast, other hormones such as e.g. glucagon and epinephrine act antagonistically and facilitate increased blood glucose levels. Insulin is synthesized in pancreatic β cells and secreted in response to e.g. increased levels of blood glucose.

Once secreted into the bloodstream, insulin acts in several processes to promote i) uptake of metabolizable substrates into certain cells, ii) storage of lipids and glycogen, and iii) biosynthesis of macromolecules such as nucleic acids and proteins. More specifically, the action of insulin results in i) an increased uptake of glucose in muscles and adipose tissues, ii) activation of the glycolytic pathway in the liver, iii) an increased synthesis of fatty acids and triglycerides in the liver and adipose tissues, iv) inhibition of lipolysis, v) inhibition of gluconeogenesis in liver, vi) an increased glycogen synthesis in liver and muscle tissue, vii) stimulation of amino acid uptake, viii) an increased protein synthesis in muscles, and ix) inhibition of proteolysis.

The blood glucose elevation occurring shortly after an intake of a meal rich in carbohydrates stimulates the secretion of insulin and concomitantly suppresses the secretion of glucagon. The combined effect thereof is a promotion of uptake of glucose into the liver, stimulation of glycogen synthesis and suppression of glycogen breakdown. When blood glucose levels subsequently begin to fall, the above events are reversed. A decreased secretion of insulin and an increased secretion of glucagon lead to the breakdown of glycogen in the liver and triglycerides in adipocytes. Triglycerides are converted into fatty acids that are used by hepatic and muscle tissues. At the same time, the decreased insulin levels serve to reduce glucose utilization by hepatic, muscle and adipose tissues.

Type 2 diabetes accounts for approximately 80-90% of all diabetes cases and is arguably the fastest growing global threat to public health. Left unchecked, the current trend has been estimated to result in 215 million sufferers from type 2 diabetes world-wide by the year 2010.

Various clinical studies have implicated obesity as a risk factor for type 2 diabetes although the underlying mechanism for its role in the pathogenesis of the disease is still unclear. Obesity amongst people, who subsequently develop type 2 diabetes, as well as those with existing type 2 diabetes, is associated with an increased hepatic output and reduced glucose utilization by peripheral tissues, such as e.g. muscles. Fatty acid metabolism is increased in both obesity and in type 2 diabetes, and this may affect glucose utilization by interfering with the actions of insulin.

The development of type 2 diabetes is progressive and likely to be a culmination of pathophysiological changes occurring over many years. In most cases, the subject is unaware of the disease process, particularly in the early stages. The first stage of the disease is thought to be initiated due to a resistance to insulin. Insulin resistance is strongly associated with, and probably a major contributor to, the disease eventually entering the diabetic state. The insulin resistance stage is characterized by reduced sensitivity to insulin, as the cells normally stimulated by insulin are less sensitive to the hormone. The next stage of the disease is that of impaired glucose tolerance (IGT). IGT follows from a continued increase in insulin resistance, i.e. a continued decrease in insulin sensitivity. Impaired glucose tolerance is formally defined as a fasting venous plasma glucose concentration <7.0 mmol/1 (126 mg/dl) and a two-hour venous plasma value after 75 gram oral glucose intake ≧7.8 (≧140) and <11,1 mmol/l (<200 mg/dl). When glucose concentration ≧11.1 mmol/1, i.e. a level indicative of type 2 diabetes, the risk of developing specific diabetic complications is greatly enhanced. However, IGT and type 2 diabetes are both associated with a 2-4 fold increase in the burden of cardiovascular diseases. The World Health Organization (WHO) glycaemic criteria have been applied in a number of studies of IGT. These studies have determined the rate of progression to type 2 diabetes—5 to 10 years after detection of IGT—at between 19% and 61%.

The final phase of type 2 diabetes development is characterized by insulin secretory failure (ISF). In this stage of type 2 diabetes, the insulin secretory response is inadequate. It is believed that this results from impairment of the pancreatic β-cell functions and/or the inability of β-cells to secrete sufficient amounts of insulin to compensate for the increased insulin resistance. It is to be understood that all of the above mentioned phases in the development of type 2 diabetes leading to overt diabetes will be considered as being comprised by the term type 2 diabetes as used herein. Accordingly, a diagnosis of impaired glucose tolerance and/or reduced insulin sensitivity will be understood to relate to an individual also diagnosed as suffering from type 2 diabetes, or a condition, including any precondition, leading to type 2 diabetes.

The progression from normal glucose tolerance (NGT) to impaired glucose tolerance (IGT) is characterized by i) an increasing insulin resistance or a decreasing insulin sensitivity and ii) gradual increases in both fasting and glucose-stimulated plasma insulin levels. As IGT gradually progresses to a mild fasting hyperglycemia, there may be a further small increase in insulin resistance in both fasting and glucose-stimulated insulin release. However, in this situation, further increases in the rate of insulin secretion are no longer sustained and overt fasting hyperglycemia and increased post-load glucose intolerance start to emerge.

As already mentioned, many cardiovascular risk factors are abnormally high in individuals suffering from type 2 diabetes and these individuals are consequently at an increased risk of developing various arteriosclerotic vascular diseases. There is at present no simple cure or medical treatment for arteriosclerosis and no effective preventive therapy for treating diabetics suffering from the symptoms of cardiovascular diseases exists.

The effect of the development of arteriosclerosis in diabetes is very clear. The proportion of total deaths from coronary heart disease (CHD) in diabetes has progressively increased and is now reported to cause almost 75 percent of all deaths among type 2 diabetics. However, even though the association between diabetes, insulin resistance, blood lipids, hypertension and premature arteriosclerosis have long been recognized, the complex mechanisms responsible for this association are still as vague and evasive as they were more than 50 years ago.

For type 2 diabetes there has actually not occurred a medical breakthrough since i) insulin was discovered in 1922 and since ii) sulphonylureas, biguanides, and α-glucosidase inhibitors were developed in 1954, 1957 and 1986, respectively. Obesity and insufficient physical exercise have been suggested to be major contributors to type 2 diabetes, and coronary heart diseases and heart infarction are among the most common cardiovascular conditions diagnosed in diabetic subjects. It has been estimated that two out of every three diabetics contract a cardiovascular disease.

An increased serum level of triglycerides is now regarded as a prominent risk factor for the development of a cardiovascular diseases, also in diabetic subjects. Importantly, recent studies have indicated that serum levels of triglycerides currently considered as “normal”—(2.2 mmol/l) or 200 mg per deciliter of serum—may in fact be too high. It has been proposed that the “normal” limit for triglycerides should be reduced by as much as 50 percent as compared to the limit presently regarded as being the “normal” limit (Yahoo News, May 1, 1998). In a group of patients examined over almost 20 years, a serum triglyceride level of more than 1.1 mmol/l or 100 mg per deciliter serum actually increased the relative risk of contracting a new cardiovascular event by 50 percent and reduced the chance of surviving that event. It was emphasized that so far no clinical trials have examined whether lowering triglyceride levels affects the incidence of subsequent cardiovascular events, and research into the effect of serum triglyceride levels on cardiovascular events lags far behind research directed to establishing the effect of increased cholesterol serum levels on the subsequent development of cardiovascular diseases.

Pulmonary diseases are diseases generally affecting the lungs. The airways and the lungs are subject to many disease causing and/or disease stimulating factors such as e.g. inhaled pathogens like bacteria and viruses, allergens and toxic substances such as cigarette smoke or air pollutants. Such factors generate disorders with symptoms like e.g. difficulty in breathing, chest pains, coughing, and wheezing.

The airways of the human and animal body consist of a series of tubes and passages that include the throat, the larynx and the trachea. In the chest cavity the trachea divides into the right and left bronchi, or bronchial tubes, that enter the lungs. The branches of the bronchi subsequently become more narrow and form tubes, the bronchioles, that divide into even more narrow tubes, the alveolar ducts. The end of each alveolar duct forms a cluster of thinly walled sacs termed the alveoli.

Several terms have been used to describe a group of conditions now generally recognized as leading to a limitation or obstruction of the flow of air in the airways and in the lungs. Obstructive pulmonary disease (OPD) and chronic obstructive pulmonary disease (COPD) are clinical terms describing diseases characterized by an obstruction or limitation of airflow during expiration. For COPD the obstruction or limitation is persistent. The terms represent a clinical rather than a pathological diagnosis and relate to diseases such as e.g. inflammation of the airways, asthma, bronchitis, and small airways diseases. However, the nomenclature in the field of obstructive pulmonary diseases is complex and sometimes confusing in spite of many attempts to define conditions such as asthma and bronchitis.

It is widely recognized that COPD is not a disease entity, but rather a complex of conditions characterized by airflow limitation or obstruction. The limitation or obstruction may be variable over short periods of time and reversible, even though an underlying irreversible trait may persist. Unless treated, the disease is likely to progress and lead to a seriously reduced airflow limitation. This reduction is usually, but not always, persistent and typically shows a more rapid progressive deterioration with age than normal. Clinical studies of acute exacerbations of obstructive pulmonary diseases are difficult because of i) the heterogeneous nature of COPD, ii) diffuse symptoms that can vary spontaneously, and iii) difficulties in defining a clinical response both in the short term and in the long run. Also, the role of e.g. bacterial infections and the subsequent use of antibiotics in connection with pulmonary diseases is controversial, and much evidence shows that although bacterial infections have a significant role in acute exacerbation, the role of said infections in the progression of obstructive pulmonary diseases is less certain.

Accordingly, any of the above-mentioned conditions—whether transient or chronic—may result in an airflow limitation or obstruction and may therefore be potentially associated with obstructive pulmonary diseases. The conditions may, however, also be present anatomically without generating an impairment of pulmonary function that is sufficient to qualify for the definition OPD or COPD.

An obstruction of the airways is measured by FEV₁ as forced expiratory volume in the first second of expiration. Lung function measured as the FEV₁ increases into young adulthood and then it starts to decrease. In normal non-smokers, the rate of decline in FEV₁ is about 20 ml per year, i.e. about 1 liter over a 50-year period. A much more rapid decline is observed in smokers. On average, the decline is twice that of normal non-smokers. However, in about 15% of all smokers, lung function declines at a rate much more rapid than the decline observed in the average smoker. Consequently, airways diseases are strongly influenced by individual rates of decline in FEV₁.

Asthma has traditionally been regarded as a respiratory disease of acute airway obstruction, and research as well as therapeutic attention has focused principally on the mechanisms leading to acute bronchospasm. One of the conventional therapies has consisted of bronchodilators to regulate airway smooth muscle contraction.

However, current state of the art asthma therapy does have side effects, mostly due to undesirable effects from the inhalation steroids used.

A wide range of pharmaceuticals have been developed by the pharmaceutical industry and evaluated in clinical trials. Although being capable of inhibiting mast cell-mediated acute allergic bronchoconstriction, none of these pharmaceuticals are suitable for use in a prophylactical treatment or maintenance treatment of asthma. Medicaments such as β₂ agonists have been introduced in order to treat airways diseases and in particular asthma. β₂ agonists inhibit the release of histamine into the circulation of asthmatics undergoing an allergen provocation. This pharmacological property may contribute to the well-recognized ability of β₂ agonists to inhibit allergen-induced bronchoconstriction. However, while β₂ agonists are exceptional mast cell stabilizing agents, sole therapy with these agents may actually enhance hyperresponsiveness of airways to exogenous stimuli such as inhaled histamine, most likely due to a minimal effect on airway inflammation.

Widespread use of β₂ agonists have lead to a criticism based on a hypothesis involving the so-called “asthma paradox”. According to the hypothesis, β₂ agonists have undesirable effects on the normal role of mast cell degranulation as an endogenous anti-inflammatory mechanism to prevent antigens from entering the lower airways and limit the extent of the subsequent repair process.

Adlercreutz (Finnish Medical Society, Ann. Med. 29, 95-120 (1997)) has reviewed the phytoestrogen classes of lignans and isoflavones and has described their influences on a range of cellular activities and metabolic events. Soy intake is reported to prevent oxidation of LDL, but no antioxidant mechanism has yet been established. Although isoflavonoids may prevent the development of atherosclerosis, it is a problem to separate the phytoestrogen effect from the effect of other components in foods. It is emphasized that phytoestrogens, particularly in association with soy intake, seem to have a great potential for preventing cardiovascular diseases, but as the area is really in the early stages of development, an established beneficial effect of soy and isoflavonoids in this respect will have to await further studies. It is stated that despite an abundant literature at this early stage of dietary phytoestrogen research, much work is needed before any recommendation as to phytoestrogen consumption can be made. However, experimental and epidemiological evidence does support the view that these compounds do not have any negative effects and that they may form a group of substances with a great potential in preventive medicine. It is emphasized that at present, no definite recommendations can be made as to the dietary amounts needed for disease prevention. No reference is made to a composition comprising a combination of soy protein, a high content of a phytoestrogen compound, and dietary fibers.

Anderson (N. Eng. J. Med. 333, 276-282 (1995)) analysed a total of 38 clinical trials and concluded that the consumption of soy protein significantly decreases serum levels of total cholesterol, LDL-cholesterol and triglycerides. It was found that ingestion of diets containing soy protein, as compared with control diets, was accompanied by a significant reduction in serum concentrations of total cholesterol, LDL-cholesterol and triglycerides. However, soy protein intake did not significantly affect serum HDL-cholesterol concentrations. The effect of soy protein intake was dependent upon initial cholesterol concentration. Subjects with normal cholesterol levels had non-significant reductions of 3.3 percent, and also subjects with mild hypercholesterolemia had non-significant reductions of 4.4 percent. Only subjects with moderate and severe hypercholesterolemia had significant decreases in cholesterol levels of 7.4 percent and 19.6 percent, respectively. The pattern of changes in serum LDL-cholesterol concentrations was similar to the pattern for total cholesterol concentrations. Also changes in serum triglyceride concentrations were significantly related to the initial serum triglyceride concentrations. Various types of soy proteins were studied, such as isolated soy protein, textured soy protein, or a combination thereof, and it was found that the type of soy protein did not have any significant effect on the net change in serum cholesterol levels. The study did not consider a simultaneous intake of the various types of soy proteins along with dietary fibers. No reference is made to a composition comprising a combination of soy protein, a high content of a phytoestrogen compound, and dietary fibers.

Bakhit (J. Nutr. 124, 213-222 (1993)) studied mildly hypercholesterolemic men receiving a baseline diet and reported that adding of 25 g of soybean protein to a low-fat, low-cholesterol diet lowers total cholesterol concentrations in men with elevated blood lipids. In subjects having lower blood cholesterol concentrations (<5.7 mmol/l), this level of soybean protein intake did not influence blood lipids, and it was suggested that plasma lipids may even be elevated in some subjects following soybean ingestion. Also, other studies have found that in general, individuals with pre-existing hypercholesterolemia respond to soybean protein, whereas individuals with normal cholesterol values do not. Bakhit et al. did not observe an additive effect of concurrent ingestion of soybean protein and soybean fiber. No reference is made to a composition comprising a combination of soy protein, a high content of a phytoestrogen compound, and dietary fibers.

Faggiotto (Atherosclerosis Reviews 21, 187-194 (1990)) states that atherosclerosis is an extremely complex disease involving different pathological processes such as inflammation and degeneration. The onset of atherosclerosis and its progression are very subtle, slow and silent processes, often overlapping with a normal aging process. It is stressed that despite a tremendous quantity of accumulated information, it is not possible to fully explain why atherosclerosis is so common in Western civilization, how fatty streaks develop in young people, how fatty streaks are converted into fibrous plaques, and what the role is of inflammation in e.g. atherosclerosis. It is stated that even when atherosclerosis becomes symptomatic, the treatment of choice often resorts to surgical procedures, as medical intervention has little or no short-term usefulness, unless patients are subjected to a relatively long-term and aggressive therapy. No reference is made to a composition comprising a combination of soy protein, a high content of a phytoestrogen compound, and dietary fibers.

Gooderham (J. Nutr. 126(8), 2000-2006 (1996)) has suggested that although soy protein supplementation to a typical Western diet may indeed increase plasma concentrations of isoflavones, this may not necessarily be sufficient to counter heart disease risk factors such as high serum levels of cholesterol and triglycerides, and platelet aggregation. Any increase in serum levels of isoflavones following intake of a soy rich diet was found to be quite variable among analysed subjects. This was thought to be due to e.g. the timing of the soy protein consumption or the composition of the gut flora. The metabolism of isoflavones in the gut is variable among individuals and remains to be elucidated. It is noted that the levels of isoflavones present in human plasma are most likely not sufficient to mediate a significant inhibition of platelet aggregation. It is stressed that the isoflavones in human plasma predominantly exist in the inactive glucuronide conjugated form, and only a small amount such as approx. 10 percent exists in the active free and sulphate conjugated forms. A lack of an effect of isoflavones on total cholesterol levels in one study was reported to be in agreement with others which also found that soy had little effect in normocholesterolemic individuals, whereas hypercholesterolemic subjects generally exhibited a decreased total and LDL-cholesterol level relative to normocholesterolemic subjects. It was stressed that only a few studies have reported an HDL-cholesterol raising effect due to the consumption of soy protein and that most studies have shown little or no effect on HDL-cholesterol levels. The reported results indicate a similar lack of effect of soy protein on HDL-cholesterol levels in normocholesterolemic subjects. It is emphasized that only recently have isoflavones been examined separately to determine if these compounds are responsible for the lipid lowering effects associated with intake of soy proteins. The administration of purified isoflavones to animals has shown variable results on blood lipids. One study conducted on hypercholesterolemic humans failed to show an effect of purified isoflavones on blood lipid levels. No reference is made to a composition comprising a combination of soy protein, a high content of a phytoestrogen compound, and dietary fibers.

Hendrich (J. Nutr. 124(9 Suppl.), 1789S-1792S (1994)) has reported that isoflavones may be of great potential benefit to human health maintenance and that isoflavones may be health-protective in amounts potentially available from a human diet containing daily soy foods. The food content of isoflavones is in the range of from 0.1 to 1 mg/g in soy foods. Several factors such as variety of soybean, processing and the addition of other ingredients to the food influence isoflavone contents of foods. It is stated that human intestinal bacteria can destroy ingested isoflavones to a great extent and that this may be why only 15 to 20 percent of isoflavones are reported to be recoverable in intact form from the urine and feces. It is emphasized that much work remains to determine the relation between concentration of isoflavones in human urine and plasma and the biological effects of the isoflavones. It is noted that although more health-related animal data need to be obtained, the time is approaching when long-term human feeding trials of purified isoflavones and foods containing isoflavones to examine health-related outcomes may be warranted. No reference is made to a composition comprising a combination of soy protein, a high content of a phytoestrogen compound, and dietary fibers.

Hunninghake (Am J. Med. 97, 504-508 (1994)) discloses an evaluation of hypocholesterolemic effects of a long-term treatment with a mixture of dietary fibers administered twice a day. 59 subjects with moderate hypercholesterolemia who completed the 15-week placebo-controlled study with the dietary fiber were treated for an additional 36 weeks with 20 g/day of fiber. There were no significant effects on the levels of either triglycerides or HDL. Levels of total cholesterol and LDL-cholesterol and the HDL/LDL-ratio were significantly reduced during treatment. The mean percentage reductions from baseline after 51 weeks of treatment were approximately 5 percent for total cholesterol, 9 percent for LDL-cholesterol, and 11 percent for the LDL/HDL ratio. No reference is made to a composition comprising a combination of soy protein, a high content of a phytoestrogen compound, and dietary fibers.

Knight (Maturitas 22, 167-175 (1995)) provides a synopsis of the literature relating principally to the clinical effects of phytoestrogens on the diseases associated with aging. A review of literature pertaining to cardiovascular diseases states that the protective effects of phytoestrogens are manifested through lipid changes, a decrease in LDL-cholesterol and an increase in HDL-cholesterol, and vascular effects, concerning both vasomotor tone and vessel wall compliance. The consumption of soy protein is reported to alter lipid levels and dietary soy protein appears to be anti-atherogenic when compared with various animal proteins. It is concluded that isoflavones represent a large and exciting group of compounds with potential benefits to many diseases, also diseases in diabetes. It is emphasized that current evidence justifies the conclusion that phytoestrogens may be among the dietary factors affording protective effects against heart disease. However, further clinical studies are required to more clearly elucidate their effects. No reference is made to a composition comprising a combination of soy protein, a high content of a phytoestrogen compound, and dietary fibers.

Knight (Obstet. Gynecol. 87, 897-904 (1996)) has reviewed the sources, metabolism, potencies, and clinical effects of phytoestrogens on humans. The review suggests that phytoestrogens are among the dietary factors affording protection against heart disease in vegetarians. Based on epidemiological and cell line studies, it is emphasized that intervention studies are now an appropriate consideration to assess the clinical effects of phytoestrogens because of the potentially important health benefits associated with the consumption of foods containing these compounds. It is concluded that clinical applications for phytoestrogens are still in their infancy. No reference is made to a composition comprising a combination of soy protein, a high content of a phytoestrogen compound, and dietary fibers.

Packard (Arterioscler. Thromb. Vasc. Biol. 17, 3542-3556 (1997)) has reviewed the heterogeneity in the apoB containing lipoprotein classes and provides an interpretation of kinetic studies of apoB metabolism in the light of underlying structural and functional variations. The review is based on the fact that lipoprotein classes are composed of a limited number of components with distinct properties. However, the basis for this heterogeneity and the consequences for disease are not thoroughly understood. The LDL-fraction is made up of a small number of subtypes of particles with relatively discrete size and density. Subjects with a preponderance of small-sized LDL have a three-fold increased risk of having a myocardial infarction independent of the total concentration of LDL present. No reference is made to a composition comprising a combination of soy protein, a high content of a phytoestrogen compound, and dietary fibers.

Potter (Am. J. Clin. Nutr. 58, 501-506 (1993)) studied the effects of soy protein consumption with and without soy fiber on plasma lipids in mildly hypercholesterolemic men. It was reported that total and LDL-cholesterol concentrations can be lowered significantly in mildly hypercholesterolemic men, as indicated by a replacement of 50 percent of dietary protein with soy protein. Similar reductions in blood lipids were noted for isolated soy protein, whether it was consumed in conjunction with soy cotyledon fiber or cellulose fiber. Plasma triglyceride concentrations were unaffected by the various dietary treatments described in the article. The study did not reveal any additive cholesterol lowering effect of concurrent intake of cotyledon soy fiber with isolated soy protein, and it was stated that whether or not there is an added benefit in lowering blood cholesterol concentrations from increased concurrent intake of soy protein and fiber in humans is not known. No reference is made to a composition comprising a combination of soy protein, a high content of a phytoestrogen compound, and dietary fibers.

Reinli (Nutr. Cancer 26, 123-148 (1996)) has reviewed the literature for quantitative data on the levels of known phytoestrogens (daidzein, genistein, coumestrol, formononetin and biochanin A) in food plants. It is reported that the isoflavones daidzein and genistein may exist in four related chemical structures, i.e. an aglycone structure (daidzein and genistein), a 7-O-glucoside structure (daidzin and genistin), a 6′-O-acetylglucoside structure (6′-O-acetyldaidzin and 6′-O-acetylgenistin), and a 6′-O-malonylglucoside structure (6′-O-malonyldaidzin and 6′-O-malonylgenistin). The conjugates (7-O-glucosides, 6′-O-acetylglucosides, and 6′-O-malonylglucosides) are transformed to aglycones, which are sometimes called free isoflavones, through hydrolysis in the intestinal tract by β-glucosidase enzymes of gut bacteria. Acid hydrolysis in the stomach may also contribute to the formation of free isoflavones. It is unclear how readily conjugates undergo intestinal hydrolysis and subsequent absorption. It is stressed that isoflavones are metabolized differently by different animals and humans. No reference is made to a composition comprising a combination of soy protein, a high content of a phytoestrogen compound, and dietary fibers.

Sniderman (Am J. Cardiol. 79, 64-67 (1997)) presents a risk factor hypothesis with an emphasis on the integral role of LDL in atherogenesis. It is stressed that a measurement of LDL-cholesterol is an incomplete estimate of the risk attributable to LDL and that other classic risk factors such as e.g. hypertension, diabetes, and smoking exert their proatherogenic potential largely or exclusively by multiplying the malign influences of LDL on the arterial wall. It is acknowledged that small, dense LDL particles are one of the most common dyslipoproteinemias associated with coronary artery disease. It is reported that elevated levels of lipoprotein (a) are associated with increased coronary risk, but the basis for this is still not clear. No reference is made to a composition comprising a combination of soy protein, a high content of a phytoestrogen compound, and dietary fibers.

WO 95/10512 relates to a process for producing an aglucone isoflavone enriched vegetable protein whey and discloses in one embodiment a whey having a dry basis genistein content of about 2.6 to about 8.7 mg/gram and a dry basis daidzein content of about 2.5 to about 6.0 mg/gram. No reference is made to a treatment of diabetes by using a composition comprising a combination of soy protein, a high content of a phytoestrogen compound, and dietary fibers. No reference is made to a treatment of a pulmonary disease by using a composition comprising a combination of soy protein, a high content of a phytoestrogen compound, and dietary fibers. No reference is made to a composition comprising a combination of soy protein, a high content of a phytoestrogen compound, and dietary fibers.

WO 95/10529 relates to a process for producing an aglucone isoflavone enriched protein concentrate and discloses in one embodiment a concentrate having on a dry basis a genistein content of about 1.0 to about 2.0 mg/gram and a daidzein content of about 0.7 to about 1.5 mg/gram. No reference is made to a treatment of diabetes by using a composition comprising a combination of soy protein, a high content of a phytoestrogen compound, and dietary fibers. No reference is made to a treatment of a pulmonary disease by using a composition comprising a combination of soy protein, a high content of a phytoestrogen compound and dietary fibers. No reference is made to a composition comprising a combination of soy protein, a high content of a phytoestrogen compound, and dietary fibers.

WO 95/10530 relates to a process for producing an aqueous extract comprising protein and glucone isoflavones and discloses in one embodiment a vegetable protein isolate having a dry basis genistein content of about 1.5 to about 3.5 mg/gram and a dry basis daidzein content of about 1.0 to about 3.0 mg/gram. No reference is made to a treatment of diabetes by using a composition comprising a combination of soy protein, a high content of a phytoestrogen compound, and dietary fibers. No reference is made to a treatment of a pulmonary disease by using a composition comprising a combination of soy protein, a high content of a phytoestrogen compound, and dietary fibers. No reference is made to a composition comprising a combination of soy protein, a high content of a phytoestrogen compound, and dietary fibers.

WO 97/31546 discloses data from total replacement programmes (for 6 weeks) in weight reduction studies conducted at Karolinska Hospital in Sweden. It is shown that products comprising isolated soy protein and soybean cotyledon fibers lower serum triglyceride levels by a maximum of 44 percent and cholesterol levels by a maximum of 27 percent for a patient population with a mean initial cholesterol content of 5.6 mmol/l. A mean value of 6.25 mmol/l was determined for all patients having serum cholesterol levels above 6 mmol/l, and for this group of patients a reduction in serum cholesterol levels of 33 percent was observed. Since the reported data were part of a weight reduction programme, a dietary effect and/or an effect related to a weight loss would have contributed to the observed reductions in cholesterol and/or triglycerides. No reference is made to a treatment of diabetes by using a composition comprising a combination of soy protein, a high content of a phytoestrogen compound, and dietary fibers. No reference is made to a treatment of a pulmonary disease by using a composition comprising a combination of soy protein, a high content of a phytoestrogen compound, and dietary fibers. No reference is made to a composition comprising a combination of soy protein, a high content of a phytoestrogen compound, and dietary fibers.

WO 97/37547 discloses an isoflavone-enriched soy protein product having a protein content greater than 60 percent of total dry matter, a total dietary fiber content of less than 4 percent of total dry matter, a sucrose content greater than 10 percent of total dry matter, a total content of sulphur-containing amino acids greater than 2.2 percent of the total amino acid content, a stachyose content of less than 1.5 percent of total dry matter, and a total isoflavone content greater than 2.5 mg/gram, equivalent to 0.25 percent. The use of soy cotyledon fibers is not anticipated and the claimed invention is for use as an ingredient in the production-of an edible product and not in a treatment of arteriosclerosis or diabetes or pulmonary diseases. Also, the product differs from the composition according to the present invention by comprising total dietary fiber in an amount of less than 4 percent of total dry matter, and by containing an unusually low amount of stachyose and a high amount of sulphur-containing amino acids. No reference is made to a treatment of diabetes by using a composition comprising a combination of soy protein, a high content of a phytoestrogen compound, and dietary fibers. No reference is made to a treatment of a pulmonary disease by using a composition comprising a combination of soy protein, a high content of a phytoestrogen compound, and dietary fibers. No reference is made to a composition comprising a combination of soy protein, a high content of a phytoestrogen compound, and dietary fibers.

U.S. Pat. No. 5,320,949 discloses a process for producing an aglucone isoflavone enriched fiber product from a vegetable protein material in the form of a slurry and discloses in one embodiment an aglucone enriched fiber product directly obtainable from said process and having a genistein content of about 1.0 and 2.0 mg/gram and a daidzein content of about 0.7 to 1.7 mg/gram. No reference is made to a treatment of diabetes by using a composition comprising a combination of soy protein, a high content of a phytoestrogen compound, and dietary fibers. No reference is made to a treatment of a pulmonary disease by using a composition comprising a combination of soy protein, a high content of a phytoestrogen compound, and dietary fibers. No reference is made to a composition comprising a combination of soy protein, a high content of a phytoestrogen compound, and dietary fibers. No reference is made to a composition comprising soy cotyledon fibers and a phytoestrogen compound.

U.S. Pat. No. 5,352,384 discloses an aglucone enriched fiber product having a genistein content of about 1.0 to 2.0 mg/gram and a daidzein content of about 0.7 to 1.7 mg/gram. No reference is made to a treatment of diabetes by using a composition comprising a combination of soy protein, a high content of a phytoestrogen compound, and dietary fibers. No reference is made to a treatment of a pulmonary disease by using a composition comprising a combination of soy protein, a high content of a phytoestrogen compound, and dietary fibers. No reference is made to a composition comprising a combination of soy protein, a high content of a phytoestrogen compound, and dietary fibers.

EP 827 698 A2 and EP 827 698 A3 disclose a process for producing an aglucone isoflavone enriched extract from a vegetable material containing isoflavone conjugates and protein. No reference is made to a treatment of diabetes by using a composition comprising a combination of soy protein, a high content of a phytoestrogen compound, and dietary fibers. No reference is made to a treatment of a pulmonary disease by using a composition comprising a combination of soy protein, a high content of a phytoestrogen compound and dietary fibers. No reference is made to a composition comprising a combination of soy protein, a high content of a phytoestrogen compound, and dietary fibers.

An abstract presented at the American Heart Association's 38^{th} Annual Conference on Cardiovascular Disease Epidemiology and Prevention held in March 1998 disclosed a reduction in the levels of total and LDL-cholesterol in a subject following intake of a diet supplemented with 25 grams of soy protein containing 4 mg, 25 mg, 42 mg, and 58 mg of isoflavones, respectively. A “dose-response” effect was reported so that increasing amounts of isoflavones were associated with an increasing reduction of cholesterol. A maximum reduction of serum levels of total cholesterol and LDL-cholesterol of 4 percent and 7 percent, respectively, was reported for the product containing 58 mg of isoflavone. No reference is made to a treatment of diabetes by using a composition comprising a combination of soy protein, a high content of a phytoestrogen compound, and dietary fibers. No reference is made to a treatment of a pulmonary disease by using a composition comprising a combination of soy protein, a high content of a phytoestrogen compound and dietary fibers. No reference is made to a composition comprising a combination of soy protein, a high content of a phytoestrogen compound, and dietary fibers.

### SUMMARY OF THE INVENTION

The present invention provides a nutritional composition having a high, fixed amount of a phytoestrogen compound such as e.g. naturally occurring isoflavones. More particularly the present invention provides a nutritional composition of soybean extractable ingredients having a high, fixed amount of a phytoestrogen compound such as e.g. naturally occurring isoflavones.

The present invention provides a combination comprising a) soy protein, preferably isolated soy protein, b) a high content of a plant hormone in the form of a phytoestrogen compound, preferably naturally occurring isoflavones, and c) dietary fibers, preferably soybean fibers, more preferably soybean fibers manufactured from the cotyledon of soybeans hereinafter referred to as soy cotyledon fibers and the present invention furthermore represents a potential new breakthrough in the treatment of cardiovascular diseases, diabetes and pulmonary diseases.

The present invention is useful in treating including prophylactically treating cardiovascular diseases such as hypercholesterolemia, hypertriglyceridemia, hyperlipidemia and other cardiovascular diseases such as e.g. arteriosclerosis. It is one objective of the present invention to significantly lower levels of total serum cholesterol and LDL-cholesterol and triglycerides in a mildly hypercholesterolemic subject. It is another objective of the present invention to significantly lower serum levels of total cholesterol and/or LDL-cholesterol and/or triglycerides in a subject suffering from hypercholesterolemia and/or hyperlipidemia. It is another objective of the present invention to render the arterial wall more resistant to the accumulation of lipoproteins. It is a further objective of the present invention to provide a composition effective in preventing, treating, prophylactically treating and/or alleviating an arteriosclerotic condition by reducing the influx of cholesterol and/or triglycerides into the endocelium of the arterial wall. Yet another objective of the present invention is to reduce lipid plaque formation.

The present invention is also useful in the prevention and/or treatment of type 2 diabetes and/or a cardiovascular disease in diabetic subjects. Accordingly, it is an objective of the present invention to effectively lower serum levels of both glucose and cholesterol and/or triglycerides. No treatment is currently available for concomitantly lowering serum levels of glucose as well as lipid serum levels of total cholesterol and/or LDL-cholesterol and/or triglycerides. It is to be understood that diabetic subjects according to the present invention have a fasting plasma glucose ≧7.0 mmol/l.

A composition according to the present invention represents a new approach to treatment of type 2 diabetes and is believed to be capable of i) lowering serum levels of glucose, ii) lowering serum levels of insulin, iii) lowering total serum levels of cholesterol and/or LDL-cholesterol and/or triglycerides, iv) increasing glucose tolerance and/or insulin sensitivity and/or v) preventing, treating and/or alleviating impaired glucose tolerance and/or insulin secretory failure in diabetic subjects and/or vi) preventing, treating and/or alleviating an arteriosclerotic condition by reducing the influx of cholesterol and/or triglycerides into the endocelium of the arterial wall of a diabetic subject suffering from a cardiovascular disease. No other known compositions are effective in lowering serum levels of both lipids and glucose and/or reducing the influx of lipids such as e.g. cholesterol and/or triglycerides into the arterial wall.

The present invention is also useful in the prevention and/or effective treatment of pulmonary diseases such as e.g. airway inflammation, asthma, bronchitis and small airways diseases, in particular asthma including chronic asthma such as e.g. asthma characterized by a chronic inflammatory condition. The present invention is believed to be capable of increasing FEV₁ of a subject, measured by forced expiratory volume in the first second of expiration, as well as being capable of treating, alleviating and/or eliminating in particular i) inflammation of the airways, ii) mucus hypersecretion, and iii) bronchoconstriction.

A composition according to the present invention may be comprised in a micronutrient as defined herein below.

Phytoestrogen compounds are naturally occurring plant hormones showing a structural similarity to 17β-estradiol. Phytoestrogens consist of a number of classes including isoflavones, coumestans, lignans and resorcylic acid lactones. The class of isoflavones consists of among others genistein, daidzein, equol, glycitein, biochanin A, formononetin, and O-desmethylangolesin. The isoflavones genistein and daidzein are found almost uniquely in soybeans. When present in the plant the isoflavones are mainly in a glucoside form, i.e. attached to a sugar molecule. Isoflavones in this glucoside form can be deconjugated to yield isoflavones in a so-called aglycone form, which is the biologically more active form of isoflavones and which is absorbed faster and to a greater extent in the human gut than isoflavones in the glucoside form. In vitro studies have examined the relative estrogenic effect exerted by various phytoestrogens including isoflavones. The resulting potencies as compared to estradiol (having a relative potency of 100), have been reported by Knight (Maturitas 22, 167-175 (1995)) for among others genistein (0.084) and daidzein (0.013). However, the results also showed that the estrogen receptor complexes formed by estradiol and isoflavones such as genistein and daidzein are functionally equivalent. The comparative dissociation constant of genistein for the estrogen receptor, as determined in competitive binding assays, was found to be from 100 to 10.000 times higher than that of estradiol.

The term “naturally occurring” substance as used in the present specification refers to a substance originally isolated from a natural source, such as an animal or a plant, for example a soy plant, or modified forms of such a substance. The naturally occurring substance for use in a composition according to the present invention may be included in a composition according to the present invention as part of the natural source or in any type of extract, isolate or the like thereof, or it may have been isolated from a plant source or synthesized biologically, microbiologically, or chemically or by any other means.

Soy proteins are involved in a reduction of cholesterol and triglyceride levels, they are easily digestible, and they represent an efficient sole protein source for maintaining the nitrogen balance. Soy isoflavones in high intakes further enhances this effect. Dietary fibers, such as soybean fibers, especially soy cotyledon fibers have been shown to lower total serum cholesterol levels, to improve glucose tolerance, to increase insulin sensitivity, to normalize the gastrointestinal function, and to exert no influence on the absorption of essential minerals.

Accordingly, in one aspect the present invention provides a composition comprising

(a) a soy protein source, selected from isolated soy protein, soy protein concentrate, or soy flour, said soy protein source providing an amount of soy protein, which is at least 45 weight percent of the total protein content of the composition, said total protein content providing at least 15 percent of the total energy content of the composition,

(b) at least one phytoestrogen compound in an amount of more than 0.10 weight percent of the soy protein content of the composition, and

(c) dietary fibers in an amount of more than 4 weight percent of the total weight of the composition on a dry basis.

In a more preferred aspect the present invention provides a composition comprising

(a) isolated soy protein in an amount of at least 50 weight percent of the total protein content of the composition, said total protein content providing at least 15 percent of the total energy content of the composition,

(b) at least one phytoestrogen compound in an amount of more than 0.10 weight percent of the soy protein content of the composition, and

(c) soybean fibers in an amount of more than 4 weight percent of the total weight of the composition on a dry basis.

In a most preferred aspect the present invention provides a composition comprising

(a) isolated soy protein in an amount of at least 50 weight percent of the total protein content of the composition, said total protein content providing at least 15 percent of the total energy content of the composition,

(b) at least one phytoestrogen compound in an amount of more than 0.10 weight percent of the soy protein content of the composition, and

(c) soy cotyledon fibers in an amount of more than 4 weight percent of the total weight of the composition on a dry basis.

Phytoestrogen compounds according to the present invention are defined as naturally occurring plant substances, which are either structurally or functionally similar to 17β-estradiol or generate estrogenic effects. Phytoestrogens consist of a number of classes including isoflavones, coumestans, lignans and resorcylic acid lactones. Examples of isoflavones according to the present invention are genistein, daidzein, equol, glycitein, biochanin A, formononetin, and O-desmethylangolesin. The phytoestrogen compounds of a composition according to the present invention are preferably isoflavones, more preferably genistein, daidzein, glycitein and/or equol, yet more preferably genistein and/or daidzein and even more preferably genistein. Genistein and daidzein are found almost uniquely in soybeans. A preferred composition according to the present invention may accordingly comprise a single isoflavone, such as genistein, daidzein, glycitein or equol, or it may comprise at least one isoflavone selected from the group comprising at least genistein, daidzein, glycitein and equol.

A composition according to the present invention may be capable of preventing, treating, prophylactically treating and/or alleviating an arteriosclerotic condition by reducing the accumulation of cholesterol in the arterial wall. This inhibitory effect may be mediated by the binding of naturally occurring isoflavones and/or soy peptides to an estrogen receptor or estrogen-like receptor present in the endocelium of an artery. The soy peptides are preferably provided by partial hydrolysis of soy protein.

Plasma cholesterol and triglyceride levels are usually increased in individuals treated for a cardiovascular disease and plasma triglyceride and lipoprotein levels are usually increased in individuals treated for type 2 diabetes and/or the metabolic syndrome. These increased levels, unless reduced by treatment, are likely to promote atherosclerosis and/or coronary heart disease (CHD). Beta-2-adrenergic receptors are present on many different types of cells including fat cells and cells of the arterial wall. Beta-2-adrenergic receptors are involved in the regulation of triglyceride synthesis in fat cells and according to one presently preferred hypothesis, binding of soy peptides and/or a phytoestrogen compound such as e.g. a naturally occurring isoflavone to a beta-2-adrenergic receptor present on a fat cell or in an arterial wall is effective in reducing e.g. the synthesis of triglycerides in fat cells and/or the release of triglycerides into the blood stream and/or reducing the influx of cholesterol and/or triglycerides into the arterial wall. The soy peptides are e.g. obtainable by partial hydrolysis of soy protein.

According to a preferred hypothesis, a composition according to the present invention will reduce and/or eliminate one or more of the risk factors for cardiovascular diseases. Accordingly, a composition according to the present invention may be effective in preventing, treating, prophylactically treating and/or alleviating conditions such as e.g. hypercholesterolemia, hypertriglyceridemia, hypertension and hyperglycemia. A composition according to the present invention may also be capable of reducing, preventing and/or eliminating fatty streak formation and/or fibrous plaque development and/or effective in mediating a regression of one or both of said arteriosclerotic conditions.

A composition according to the present invention may be effective in preventing and/or treating type 2 diabetes and/or the metabolic syndrome and/or reducing and/or eliminating one or more of the risk factors for cardiovascular diseases associated with diabetes and/or the metabolic syndrome. Accordingly, a composition according to the present invention may be effective in preventing, treating, prophylactically treating and/or alleviating conditions such as e.g. increased serum levels of glucose, hypercholesterolemia, hypertriglyceridemia, hypertension, and hyperinsulinemia in diabetic individuals. A composition according to the present invention may also be capable of reducing, preventing and/or eliminating fatty streak formation and/or fibrous plaque development and/or effective in mediating a regression of one or both of said arteriosclerotic conditions in diabetic individuals.

According to a preferred hypothesis, a composition according to the present invention will be effective in lowering serum levels of total cholesterol and/or LDL-cholesterol and/or triglycerides and/or in increasing the serum HDL/LDL-cholesterol ratio and/or increasing serum levels of high-density lipoproteins (HDL) and/or in generating a decrease in serum levels of low-density lipoproteins (LDL). It is desirable to achieve an elevated serum HDL/LDL-cholesterol ratio since this may result in an increased reverse cholesterol transport and a subsequent excretion.

Also, it is believed that a composition according to the present invention will affect ApoB lipoprotein metabolism including the metabolism of a recently discovered class of ApoB comprising lipoprotein particles called small, dense LDL particles. The LDL class of lipoproteins is in fact composed of several components with distinct properties. The basis for this heterogeneity and the consequences for disease are at present not thoroughly understood. An increased level of small, dense LDL particles is one of the most common dyslipoproteinemias associated with coronary artery disease, and serum levels of ApoB are often disproportionately elevated compared with LDL-cholesterol in dyslipoproteinemic patients.

Heterogeneity within lipoprotein classes may be the result of a differing lipid content, a different apoprotein composition, an altered protein conformation or an as yet unidentified structural variation. Subjects with a preponderance of small, dense LDL have an increased risk of suffering a myocardial infarction independent of the total concentration of serum LDL. Accordingly, a composition according to the present invention may be effective in lowering elevated levels of small, dense LDL.

Hypertriglyceridemia in non-diabetic and diabetic subjects alike is associated with an increase in the clotting activities of thrombogenic factors such as e.g. factor VII and/or factor X and/or factor XII and an increase in the level of the inhibitor of tissue plasminogen activator, PAI-1. The increased inhibitor concentration results in a decreased level of plasminogen synthesis and thus a decreased level of plasminogen stimulated clot lysis. These changes in clotting activities no doubt contribute to the procoagulant state, which is also observed in diabetes. Accordingly, the present invention provides a composition, which may be effective in normalizing levels of homocystein and/or the clotting activities of at least one thrombogenic factor such as e.g. factor VII and/or factor X and/or factor XII by e.g. decreasing the increased activity thereof, which is also observed in a subject diagnosed as having type 2 diabetes or diagnosed as having an impaired glucose tolerance or a decreased insulin sensitivity. Also, a composition according to the present invention may be effective in promoting a decrease in the level of the inhibitor of tissue plasminogen activator, PAI-1, which in turn leads to an increased plasminogen stimulated clot lysis. A composition according to the present invention may also be effective in reducing an increased platelet aggregatability and/or mediating directly or indirectly a reduction in the increased level of lipoprotein (a) associated with a procoagulant state in an arteriosclerotic condition and/or a diabetic condition.

Accordingly, in one embodiment the present invention provides a composition effective in reducing and/or eliminating risk factors for coronary heart disease (CHD) in obese subjects and in obese subjects suffering from a diabetic condition and/or the metabolic syndrome. Consequently, a composition according to the present invention may be capable of preventing, treating, prophylactically treating, alleviating and/or eliminating hyperinsulinemia and/or hypertriglyceridemia and/or hypercholesterolemia and/or hyperglycemia and/or hypertension and/or effective in mediating an increase in the low serum levels of HDL-cholesterol and/or effective in mediating an increased serum HDL/LDL-cholesterol ratio.

A composition according to the present invention may also be effective in treating dyslipidemia such as e.g. hypertriglyceridemia and/or hypercholesterolemia in connection with increased serum levels of VLDL, decreased and altered serum levels of HDL and increased serum levels of small dense LDL, and hypertension, all of which are risk factors for atherosclerosis. Accordingly, in one embodiment, a composition according to the present invention may be capable of effectively lowering and/or eliminating increased serum levels of VLDL, and/or effectively increasing decreased serum levels of HDL, and/or effectively lowering serum LDL levels including serum levels of small dense LDL. A composition according to the present invention may be capable of preventing, treating, prophylactically treating and/or alleviating hypertension.

A composition according to the present invention may also be effective in suppressing any effect that would otherwise generate an increased turnover of arterial smooth muscle cells, i.e. an enhanced arterial smooth muscle cell proliferation, and/or lead to an increased cholesterol ester accumulation in the arterial wall.

In hypercholesterolemia characterized by increased levels of intracellular cholesterol resulting from e.g. increased delivery of LDL-cholesterol via the LDL receptor, a composition according to the present invention may be effective in reducing the increased activity of the LDL receptor. It is also possible that insulin and other growth factors have the potential to promote the accumulation of cholesterol intracellularly. This may in fact well occur in a diabetic subject and more generally under conditions when cells are stimulated, but cannot proliferate normally. Accordingly, a composition according to the present invention may also be capable of treating, alleviating and/or eliminating any decrease, including any insulin mediated decrease, in the HDL receptor-mediated cholesterol efflux. Accordingly, a composition according to the present invention may be capable of reducing and/or eliminating any enhanced retention of intracellular cholesterol caused by a decreasing HDL receptor-mediated cholesterol efflux.

A composition according to the present invention may be effective in reducing insulin resistance by stimulating cells or receptors located thereon that are normally stimulated by insulin, but less sensitive to the hormone in a subject diagnosed with type 2 diabetes and/or the metabolic syndrome. A composition according to the present invention may also be effective in stimulating cells comprising a beta-2-adrenergic receptor or a receptor belonging to the class of beta-2-adrenergic receptors. The final phase of type 2 diabetes development is characterized by insulin secretory failure (ISF), and in one presently preferred hypothesis, this failure is at least preventable by a composition according to the present invention effective in stimulating insulin secretion.

Hypertriglyceridemia in diabetes has been associated with a variety of changes in circulating lipoproteins, and a composition according to the present invention may be capable of preventing, treating, alleviating and/or eliminating cardiovascular risk factors such as e.g. chylomicronemia, an increased level of VLDL, an increased level of remnants (VLDL and chylomicrons), and LDL and HDL containing increased levels of triglycerides.

Lipoprotein fractions obtained from type 2 diabetic subjects tend to lose their typical sharp LDL peak and instead have a broad diffuse LDL band termed polydisperse LDL. Dissection of polydisperse LDL reveals that diabetics have an increased serum level of intermediate-density-lipoprotein (IDL), an abnormal LDL peak, and an increase in the amount of small dense LDL. While small dense LDL particles have been associated with CHD in the general population, a similar association in diabetes remains to be established. Accordingly, a composition according to the present invention may be effective in promoting a decreased serum level of intermediate density lipoprotein (IDL), a normal, sharp LDL peak, and a decreased amount of small dense LDL.

Accordingly, diabetic dyslipidemia of type 2 diabetes is generally associated with abnormalities of apolipoprotein and lipoprotein particle distributions and results in increased plasma VLDL and remnant levels, an increase in the apoE concentration in VLDL and remnants, an increase in the amount of small dense LDL, and an altered HDL particle distribution.

According to one presently preferred hypothesis, a composition according to the present invention will alleviate abnormalities associated with apolipoprotein and lipoprotein particle distribution and promote a decreased plasma VLDL and remnant level, a decrease in the apoE concentration in VLDL and remnants, a decrease in the amount of small dense LDL, and a HDL particle distribution similar to that of a comparable non-diabetic, healthy individual.

Hyperinsulinemia is also considered a risk factor for coronary heart disease (CHD) in diabetic subjects due to the association of high insulin levels with increased incidence and mortality rates of CHD. A composition according to the present invention may be effective in lowering serum insulin levels in subjects diagnosed with type 2 diabetes. Diabetic patients having increased endogenous insulin levels, i.e. subjects diagnosed with type 2 diabetes, or having increased peripheral circulating insulin levels as a result of intermittent injections of large amounts of exogenous insulin are particularly prone to hyperinsulinemia.

Hyperinsulinemia in both normal persons, persons with the metabolic syndrome and those with type 2 diabetes appears to be related to obesity. Insulin levels are very often increased in both the fasted state and after intake of a diet rich in carbohydrates in obese individuals, irrespective of whether they suffer from a diabetic condition or not. Furthermore, hyperinsulinemia appears to be directly correlated to the degree of obesity. Accordingly, hyperinsulinemia is one of the many risk factors for CHD associated with obesity, and insulin may modulate many other obesity-related risk factors. Accordingly, a composition according to the present invention may be effective in lowering insulin levels in obese subjects with diabetes or the metabolic syndrome.

In obese subjects diagnosed as diabetic, LDL particle size is independently correlated with factors such as e.g. serum triglyceride and serum insulin levels. Consequently, it is possible that the extent of adiposity and concomitant insulin resistance in hyperinsulinemic individuals is associated with the occurrence small dense LDL, independently of hypertriglyceridemia, which is another diabetic condition also putatively associated with small dense LDL formation. Accordingly, both insulin resistance and hyperinsulinemia appear to play a central role in the pathogenesis of atherosclerosis in diabetes. A composition according to the present invention may be effective in treating and/or alleviating insulin resistance and/or hyperinsulinemia.

It is very possible that type 2 diabetes is also associated with insulin resistance and hyperinsulinemia independently of an increase in abdominal lipids. Hyperinsulinemia in turn is associated with dyslipidemia, i.e. increased VLDL, decreased and altered HDL and increased small dense LDL, and with hypertension, all of which are risk factors for atherosclerosis. This array of abnormalities and disorders, or a part of thereof, is generally termed the insulin resistance syndrome, or syndrome X, or metabolic syndrome.

Even though there is no internationally agreed definition for the metabolic syndrome, the term as used herein shall be understood to relate to the occurrence in a subject of at least two of the following: i) impaired glucose tolerance, ii) elevated blood pressure, iii) hypertriglyceridemia and low HDL-cholesterol, iv) insulin resistance, and v) obesity. The occurrence of a condition characterized by one or more of impaired glucose tolerance, elevated blood pressure, hypertriglyceridemia and low HDL-cholesterol, insulin resistance, and obesity will depend on variables such as sex, age, body weight, physical condition and the like, and general WHO guidelines will generally be adhered to when evaluating the occurrence of any one of the above-mentioned conditions.

In one embodiment, a composition according to the present invention may be capable of effectively decreasing and/or eliminating increased serum levels of VLDL and/or LDL, and/or increasing decreased serum levels of HDL, and of decreasing and/or eliminating serum LDL levels including serum levels of small, dense LDL. A composition according to the present invention may also be capable of reducing an elevated level of small, dense LDL particles and/or reducing an elevated ratio of LDL-apoB to LDL-cholesterol and/or preventing, treating or alleviating hypertension.

Hyperinsulinemia in itself may well be capable of affecting the arterial wall either directly or indirectly by promoting or facilitating the promotion of changes similar to those leading to severe atherogenesis. Insulin may well promote both arterial smooth muscle cell proliferation and cholesterol ester accumulation in the arterial wall. A composition according to the present invention may in one embodiment be effective in preventing, treating, alleviating and/or eliminating fatty streak formation, fibrous plaque development, complicated lesion formation, thrombosis, platelet aggregation and/or myocardial infarction.

Since insulin can be expected to be capable, either in combination with other compounds such as additional growth factors, or on its own, of increasing the levels of intracellular cholesterol, by e.g. increasing a delivery of LDL-cholesterol via the LDL receptor, and concomitantly therewith increase an endogenous biosynthesis of cholesterol that makes yet more cholesterol available for new membrane synthesis in the cell proliferation process, it is an object of the present invention to counteract any increased activity including any insulin stimulated increased activity of the LDL receptor.

Modifications of lipoproteins constitute another risk factor for cardiovascular disease, including cardiovascular disease in diabetes. The modification characterized by protein glycosylation is associated with e.g. arteriosclerosis and diabetes, and glycosylated lipoproteins such as e.g. LDL, IDL, VLDL and HDL can be expected to be functionally abnormal. Accordingly, the accumulation of glycosylated LDL in the plasma, including the plasma of a diabetic subject, can be perceived to enhance cholesterol ester accumulation. Also, glycosylation of HDL can be expected to impair the ability of HDL binding to the HDL receptor. This impaired binding is likely to reduce the level of intracellular cholesterol efflux. Accordingly, glycosylated HDL may well be another factor potentially contributing to the accumulation of cholesterol in the arterial cell wall. A composition according to the present invention may be effective in preventing, treating, alleviating, reducing and/or eliminating lipoprotein glycosylation in the serum of subjects; including diabetic subjects. In addition, a composition according to the present invention may also be effective in preventing lipoprotein modification caused e.g. by oxidation, chemical modification such as chemical cross-linking, or modifications caused by an alteration in the lipid composition of the lipoprotein, such as any increase or decrease in the content of triglycerides, cholesterol esters, free cholesterol, and apolipoproteins.

Glycosylated lipoproteins have been suggested to be the subject of further processing leading to the formation of hyperglycosylated compounds. Glycosylation and hyperglycosylation of proteins including lipoproteins in both plasma and the arterial wall can also be expected to be a risk factor for cardiovascular disease including arteriosclerosis, also in diabetic subjects. Accordingly, a composition according to the present invention may be capable of preventing, treating, alleviating, reducing and/or eliminating the accumulation of hyperglycosylated proteins in both serum and cells of the arterial wall. By doing so, the composition is acting to decrease the amount of LDL becoming “trapped” in the arterial wall due to the high degree of glycosylation of arterial wall proteins. A composition according to the present invention may also be effective in preventing and/or alleviating any change to the endothelial cell wall that increase LDL “trapping”, and it may be effective in restoring the formation of cells with normal permeability and adhesion parameters.

Lipoprotein glycosylation, hyperglycosylation, oxidation and/or auto-oxidative glycosylation, are risk factors for cardiovascular disease such as arteriosclerosis, including arteriosclerosis in diabetes. Accordingly, a composition according to the present invention may be effective in preventing, treating, alleviating, eliminating and/or reducing any incidence of lipoprotein glycosylation, hyperglycosylation, oxidation and/or auto-oxidative glycosylation. According to one presently preferred hypothesis, the phytoestrogen compound of a composition according to the present invention is capable of counteracting such incidences. The phytoestrogen compound may also be capable of preventing, reducing and/or eliminating the formation of e.g. free radicals that are likely to be involved in such processes, and a composition according to the present invention may be effective in being, promoting, and/or facilitating the formation of an effective antioxidant defense system for counteracting glycosylation, hyperglycosylation, oxidation and/or auto-oxidative glycosylation of serum proteins and proteins including lipoproteins of the arterial cell wall.

Since oxidative stress is a characteristic of diabetes-and possibly a contributory factor to among others lipoprotein oxidation and/or glycosylation, and since no efficient antioxidant protection exists due to e.g. significantly decreased levels in diabetic subjects of antioxidants such as e.g. ascorbic acid, a composition according to the present invention may be effectively acting as an antioxidant in preventing lipoprotein oxidation and/or glycosylation.

Generally, a composition according to the present invention may be effectively acting as an antioxidant in preventing lipoprotein oxidation and/or glycosylation. By the term auto-oxidative glycosylation, or glycoxidation, is understood a reaction catalyzed e.g. by reducing sugars that leads to an oxidative modification and/or cross-linking of proteins. The rate of such a process can be expected to be increased in the presence of high glucose concentrations since the oxidizing potential is significantly increased under such circumstances. An increased production of free radicals and lipid peroxidation may also contribute to the formation of auto-oxidative glycosylated lipoproteins and this contribution may also be effectively prevented and/or eliminated by a composition according to the present invention.

According to another presently preferred hypothesis, the binding of phytoestrogen compounds such as e.g. isoflavones, optionally in combination with soy peptides e.g. obtainable by partial hydrolysis of soy protein, to a receptor in the arterial wall, such as e.g. the estrogen receptor, or an estrogen-like receptor, is involved in or effective in controlling accumulation of lipoproteins and uptake of cholesterol and/or triglycerides in the arterial wall, possibly by regulating the permeability of said wall and/or the mechanism of lipoprotein and/or cholesterol and/or triglyceride transport across cellular membranes. Consequently, the binding of isoflavones such as e.g. genistein and/or daidzein to a receptor in the arterial wall may reduce the flux of lipoproteins into the arterial wall and/or prevent cholesterol and/or triglycerides from entering the arterial wall, or reduce and/or substantially eliminate the amount of cholesterol and/or triglycerides that enters the arterial wall. Receptor binding of isoflavones in the arterial wall is particularly effective in controlling, preventing and/or eliminating fatty streak formation and/or fibrous plaque development and/or effective in mediating a regression of one or both of said arteriosclerotic conditions.

According to a particularly preferred hypothesis, binding of an isoflavones such as e.g. genistein and/or daidzein to a receptor in the arterial wall, preferably an estrogen receptor or an estrogen-like receptor, results in an increased nitric oxide synthesis in the endothelial cells of the arterial wall. Nitric oxide is known to exert anti-arteriosclerotic effects including inhibition of platelet adhesion and aggregation, and inhibition of smooth muscle cell proliferation. Soy peptides obtainable by hydrolysis of soy protein may participate in the binding of isoflavones to an estrogen receptor or an estrogen-like receptor or the soy peptides may themselves bind to said receptor and exert an action leading to an increased nitric oxide synthesis.

In another presently preferred hypothesis, the establishment of an oxidative potential that promotes lipoprotein oxidation and/or lipoprotein auto-oxidative glycosylation occurs concomitantly with, and is very likely caused by, a decrease in cellular antioxidative defense systems. This hypothesis is supported by the fact that e.g. ascorbic acid concentrations are decreased in many diabetic individuals. Accordingly, a composition according to the present invention may be effective in acting as an antioxidant. This action reduces and/or eliminates LDL, VLDL, IDL and/or HDL susceptibility to oxidation. Concomitantly with a direct anti-oxidative effect, a composition according to the present invention may also lower the increased serum glucose levels and by doing so, a composition according to the present invention may be effective in reducing the oxidizing potential causing and/or contributing to oxidative stress.

Furthermore, a composition according to the present invention may also be effective in reducing an enhanced susceptibility to endothelial injury and/or for alleviating and/or restoring and/or improving an inefficient endothelial cell repair mechanism leading to endothelial dysfunction. One effect of such an action exerted by a composition according to the present invention is to direct macrophage development away from foam cell formation and to increase the potential of generating arterial smooth muscle cells.

The unique dyslipidemia associated with type 2 diabetes is a major risk factor for cardiovascular disease, and prevention, alleviation, reduction and/or elimination of dyslipidemia in diabetic subjects is a prime objective of administration of a composition according to the present invention to a diabetic individual. Another important objective of such an administration is the development in a diabetic subject of a gradually reduced insulin resistance and/or a gradually improved glucose tolerance. Since increasing insulin resistance and impaired glucose tolerance are key elements in the progression of type 2 diabetes, the same factors most also be a natural focus of any preventive treatment.

In another presently preferred hypothesis, a composition according to the present invention will promote and/or mediate a reduction in arterial wall thickness and lead to a reduction in the amount of LDL entering the wall. It is believed that an increased thickness of the arterial wall is positively associated with an increased uptake of LDL particles that are likely to either aggregate or oxidize within the cells of the arterial wall.

Also, a composition according to the present invention may be capable of reducing, eliminating and/or preventing the formation of increased serum levels of lipoprotein (a) in a subject, including a diabetic subject. Lipoprotein (a) levels may primarily be genetically determined, and no current cardiovascular medications are thought effective in lowering serum levels of lipoprotein (a).

Obstructive pulmonary disease (OPD) including chronic obstructive pulmonary disease (COPD) as used herein is defined as a condition comprising subjects with airways limitations or obstructions or subjects with a mucus hypersecretory condition including chronic mucus hypersecretion, i.e. subjects with asthma including chronic asthma and subjects with bronchitis including chronic bronchitis. However, a clear distinction between e.g. bronchial asthma and chronic bronchitis can be difficult and sometimes impossible to make, and a sharp distinction between COPD and OPD is therefore not always possible.

Mucus hypersecretion and a limited or obstructed airflow are two major characteristics of COPD. According to one presently preferred theory, mucus hypersecretion is an initial mechanism that leads to recurrent respiratory infections, that in turn generates a destruction of the airways and promotes a development of pulmonary parenchyma and airflow obstruction. At least two separate conditions, i) mucus hypersecretion and ii) dyspnea, are identifiable due to an obstructive or limited lung function. Chronic mucus hypersecretion and obstructive airflow are not necessarily related, since an individual may have a hypersecretory disorder only, or an obstructive disorder only, or both a hypersecretory and an obstructive disorder. Chronic mucus hypersecretion is associated with an impaired mucociliary clearance and may therefore predispose to lung cancer by causing a prolonged contact between potential carcinogens with the bronchial epithelium. Accordingly, a composition according to the present invention may be effective in treating and/or alleviating mucus hypersecretion and dyspnea in a subject.

Asthma as used herein is defined as a respiratory disease in which spasm and constriction of the bronchial passages and swelling of their mucous lining cause obstruction of breathing, often, but not-exclusively, due to allergy. One mechanism for expiratory airflow limitation in asthma is a smooth muscle contraction leading to a narrowing of the airway lumen. Asthma is frequently divided clinically into extrinsic and intrinsic asthma, separating asthma triggered by environmental allergens from that in which atopy does not appear to play a major role. Consequently, a composition according to the present invention may be effective in preventing, treating and/or alleviating smooth muscle contraction.

In asthma the airways are occluded by tenacious plugs of exudate and mucus, and there occurs a fragility of airway surface epithelium, thickening of the reticular layer beneath the epithelial basal lamina, bronchial vessel congestion and edema. An increased inflammatory infiltrate comprising “activated” lymphocytes and eosinophils, and an enlargement of bronchial smooth muscle, particularly in medium-sized bronchi, is also observed. Asthma comprises at least extrinsic (atopic or allergic) and intrinsic (non-atopic) divisions, each of which present clinically in a variety of ways. A composition according to the present invention may be effective in preventing and/or alleviating the formation of tenacious plugs of exudate and mucus, effective in preventing, treating and/or alleviating a fragility of airway surface epithelium subsequently generated by mucus secretion, effective in preventing, reducing and/or eliminating any thickening of the reticular layer beneath the epithelial basal lamina, and effective in preventing, treating and/or alleviating bronchial vessel congestion and/or edema.

Asthma may in some cases be regarded as a chronic inflammatory disease. Since the term chronic asthmatic bronchitis has no clearly defined pathologic equivalent, patients having a chronic productive cough normally associated with chronic bronchitis, as well as bronchospasms, at the same time as having an airflow obstruction, will be regarded as suffering from both chronic bronchitis as well as small airways disease (chronic obstructive bronchitis) and asthma, since the pathology presumably would be that of those conditions.

A composition according to the present invention may be effective in preventing, alleviating and/or curing inflammation of the airways, whether transient or chronic. Airway inflammation is thought to be an important contributor to asthma, and airway inflammation may well be present even in the absence of severe symptoms of asthma. In one particularly preferred aspect the present invention provides a treatment and/or alleviation of an inflammation of the airways by means of an anti-oxidative effect exerted by a composition according to the present invention. The anti-oxidative effect may in particular be exerted by naturally occurring isoflavones forming part of a composition according to the present invention.

A composition according to the present invention may be effective in increasing FEV₁, as measured by forced expiratory volume in the first second of expiration, said effect being exerted by the binding of a component of the composition, particularly naturally occurring isoflavones, to beta-2 receptors or receptors belonging to the class of beta-2 receptors. Beta-2 receptors are present on many different types of cells including cells in airways and vessels. A composition according to the present invention may also be effective in generating a dilatation of the airways in a subject, preferably a subject suffering from a pulmonary disease.

The occurrence of bronchial inflammation in asthma is, according to one presently preferred hypothesis, thought to arise at least in part from an airway response to an antigen in an allergic subject. The response includes immediate pulmonary mast-cell activation and initiation of an inflammatory response that develops over hours and is important in the later and more persistent development of bronchial obstruction. A composition according to the present invention may be effective in treating, alleviating and/or eliminating several of the causes of airway obstruction that—alone or in combination—contributes to bronchial hyperresponsiveness, i.e. the fundamental defect in asthma. Importantly, airway inflammation is believed to be a crucial component for i) the chronicity of asthma, ii) the intensity of airways hyperresponsiveness, and iii) the absence of a complete therapeutic control, when bronchodilator therapy is used alone. Consequently, a composition according to the present invention may be effective in controlling, reducing and/or eliminating edema, mucus secretion, and inflammation of the airways resulting at least in part from a response to an allergen.

Although the precise pathogenesis of asthma has yet to be discovered, allergic reactions and respiratory infections are particularly important. Both are frequent factors in asthma and exacerbations of asthma, and both not only trigger acute asthmatic symptoms but may also enhance the degree of airway hyperresponsiveness long after the initial stimulus has been removed. Of particular interest has been the airway's response to an inhaled antigen. Almost all subjects with allergic asthma experience immediate bronchospasm following inhalation of an antigen, i.e. acute airway obstruction, within 15 min of antigen exposure. In these subjects, antigen inhalation initiates not only immediate bronchocontraction, but also the reappearance of airway obstruction 4 to 6 hours later, a condition known as late asthmatic reaction or LAR. The late asthmatic response has a number of features that are characteristic of chronic asthma such as e.g. less responsiveness to bronchodilator therapy than the isolated acute event, an increased airway responsiveness, and the development of bronchial inflammation. Two features of the LAR to antigen inhalation suggest a linkage to the pathogenesis of asthma: The presence of bronchial inflammation and the enhancement of bronchial responsiveness. Consequently, a composition according to the present invention may be capable of preventing both immediate bronchocontraction as well as a late asthmatic reaction.

Asthmatic reactions following inhalation of an antigen include an immediate release from pulmonary mast cells of preformed mediators and a generation of a variety of factors needed to initiate an acute allergic airway reaction. Because the airways of patients with asthma are hyperresponsive, the immediate bronchial reaction to mast cell bronchospastic mediators is accentuated beyond the pharmacological properties of these substances. With cellular activation by antigen and membrane-bound IgE interaction, the mast cell initiates a generation of leukotrienes and prostaglandins. The leukotrienes, C₄, D₄, E₄, along with histamine, are undoubtedly involved in the acute bronchospastic response because of their airway smooth muscle contractile properties. The generation and release by mast cells of chemotaxic factors is important for the recruitment of inflammatory cells to the airway and for the subsequent development of the late asthmatic response. Accordingly, a composition according to the present invention may be capable of effectively reducing or eliminating mast cell mediated secretion of mediators such as e.g. heparin, histamine and sulphidopeptide leukotrienes C₄, D₄, and E₄.

Associated with the development of the LAR is a recruitment of inflammatory cells to the airway, including neutrophils, macrophages, lymphocytes, eosineophils, monocytes, and basophils. With their entry into the airways, and presumable cellular activation, airway obstruction reappears. It is thought that components of airway obstruction in LAR include bronchospasm, edema, and inflammation. An additional consequence of the LAR is an increase in airway hyperresponsiveness; thus, the asthmatic process is further perpetuated and positively reinforced. Consequently, a composition according to the present invention may be capable of effectively controlling in a late asthmatic response the symptoms of bronchospasm, edema, and inflammation, and in addition also effectively controlling such as reducing and/or eliminating any increase in airway hyperresponsiveness.

Furthermore, mast cells may according to another presently preferred hypothesis produce various cytokines, interleukin 3 (IL-3), interleukin 5 (IL-5), and granulocyte/macrophage colony-stimulating factor (GM-CSF), which can perpetuate the allergic reaction by further priming inflammatory cells. Consequently, a composition according to the present invention may be capable of effectively controlling i.e. reducing and/or eliminating the production of various cytokines, interleukins such as e.g. interleukin 3 (IL-3) and interleukin 5 (IL-5), and granulocyte/macrophage colony-stimulating factor (GM-CSF), and reduce any further priming of inflammatory cells during an early and/or late asthmatic response.

A class of cells termed neutrophils can be found in lavage fluid from an asthmatic subject, but the precise role of neotrophils in the generation of a late allergic reaction has not yet been established. The neutrophil cell has a potential for generating inflammation by releasing e.g. lysosomal enzymes, oxygen metabolites, leukotriene B₄ and by synthesizing histamine-releasing factor (HRF). HRF can amplify the allergic reaction by causing mediator release from a class of cells termed basophils that also appear during a late allergic reaction. Consequently, a composition according to the present invention may be capable of effectively controlling i.e. reducing and/or eliminating a neutrophil production of e.g. lysosomal enzymes, oxygen metabolites, leukotriene B₄ and histamine-releasing factor (HRF).

Evidence also exists for an implication of the group of cells termed eosinophils in an asthmatic response. Circulation of eosinophils leads to an increased severity of airway obstruction. Eosinophil granular associated proteins, including major basic protein (MBP), eosinophil cationic protein, eosinophil-derived neurotoxin, and eosinophil peroxidase are known to have profound effects on airway and cell function. MBP in particular has a number of unique properties accentuating the asthmatic response. MBP can directly injure airway epithelium, promote bronchial responsiveness, and mediate smooth muscle contraction. MBP further activates the release of mediators from mast cells and basophils. Eosinophils may also be involved in initiating tissue damage associated with various allergic diseases, such as e.g. the epithelial desquamation observed in asthmatics. This tissue damage has been suggested to be mediated in part via the release of cytotoxic mediators such as major basic protein (MBP), eosinophil cationic protein (ECP), and eosinophil peroxidase (EPO).

Eosinophil activation results in the release of a number of important mediators, including leukotriene C₄, which can contract airway smooth muscle, and platelet-activating factor (PAF). The release process of PAF has not been fully defined, but if secreted, this lipid mediator could contract airway smooth muscle as well as increase bronchial responsiveness. Furthermore, PAF is a potent eosinophil chemoattractant and a functional primer. Accordingly, eosinophils possess properties directly and indirectly causing airway obstruction and promoting bronchial hyperresponsiveness. Consequently, a composition according to the present invention may be capable of effectively controlling i.e. reducing and/or eliminating any increase in the formation of eosinophils during an asthmatic response. A composition according to the present invention may further be effective in controlling the production of eosinophil granular associated proteins including major basic protein (MBP), eosinophil cationic protein, eosinophil-derived neurotoxin, and eosinophil peroxidase. In an even further embodiment, a composition according to the present invention may be effective in controlling i.e. reducing and/or eliminating the release of mediators from mast cells, neutrophils, basophils and eosinophils, in particular the release of mediators such as e.g. leukotriene C₄ and platelet-activating factor (PAF), IL-3, GM-CSF and IL-5.

To generate airway inflammation after eosinophil recruitment, a number of events need to occur such as e.g. eosinophil migration to the lung and eosinophil activation. The last event is likely to involve eosinophil adhesion to endothelium and, eventually, airway epithelium. Accordingly, a composition according to the present invention may be effective in preventing eosinophil participation in the bronchial responsiveness process by inhibiting eosinophil adhesion to endothelium and epithelium.

Mast cells may also release compounds such as heparin and related proteoglycans, but the release of such mediators have so far not received much attention from allergy researchers. These highly anionic molecules are normally only associated with the binding histamine within mast cell granules. These molecules may act as natural antiinflammatory molecules and, thus, have a far greater role in the pathogenesis of allergic diseases. Accordingly, a composition according to the present invention may be effective in promoting the release of potentially antiinflammatory molecules such as e.g. heparin and related proteoglycans. Also, it has been reported that another cationic protein, platelet factor 4 (PF4), is a chemotaxic agent for human eosinophils and is a molecule well recognized for its ability to bind heparin. It is therefore plausible that endogenous heparin could be released to limit both the extent of eosinophil recruitment into sites of allergic inflammation as well as the extent of tissue damage induced by cationic proteins. Lymphocytes are also likely to be involved in the pathogenesis of allergic asthma. Recent studies have suggested that heparin acts as an immunomodulator inhibiting lymphocyte activation and trafficking and, like glucocorticosteroids, can also inhibit delayed hypersensitivity responses.

Further evidence of asthma being a chronic inflammatory disease is provided by the observation that an exposure to an allergen that results in tissue damage is likely to lead to a repair of the damage. Evidence of this repair process can most likely be seen in the asthmatic lung, where a thickened basement membrane is believed to be related to subepithelial fibrosis, the presence of myofibroblasts, and collagen deposition. Also, asthma is further characterized by a thickened smooth muscle layer. The above-mentioned changes appear very early in the disease and are not constricted to patients with chronic asthma. Furthermore, even following chronic treatment with inhaled glucocorticosteroids for periods of up 10 years, the thickness of the basement membrane is not reduced, although such therapy reduces the number of inflammatory cells present in the biopsies and the extent of the epithelial damage. Such clinical observations suggest that once these anatomic changes have appeared they may not be readily reversible, even with the most aggressive therapy currently available. Thus, it is plausible that once established, such anatomic changes may underlie the irreversible component of the disease, and by altering the geometry of the airway wall, these changes may contribute to the persistent airways hyperresponsiveness that does not respond to treatment. Accordingly, a composition according to the present invention may, in one particularly preferred embodiment, be capable of effectively preventing and/or alleviating the formation of a thickened basement membrane or a smooth muscle layer, subepithelial fibrosis, the presence of myofibroblasts, and a deposition of collagen.

Bronchitis as used herein is defined as an acute or chronic inflammation of any part of the bronchi and bronchial tubes. The bronchi are large delicate tubes in the lungs that are attached to the trachea and carry air to smaller tubes in the lungs. In bronchitis, including chronic bronchitis, there is mucous hypersecretion, an enlargement of tracheobronchial submucosal glands, and a disproportionate increase of mucous acini. Acute bronchitis is often characterized by fever, chest pain, severe coughing, and secretion of mucous material coughed up from the respiratory tract. Acute bronchitis affects the branches of the bronchi and may develop into bronchial or lobular pneumonia. Chronic bronchitis may result from repeated attacks of acute bronchitis. Consequently, a composition according to the present invention may be effective in controlling mucous hypersecretion, preventing, treating and/or alleviating an enlargement of tracheobronchial submucosal glands, and reduce and/or eliminate a disproportionate increase of mucous acini. The pathologic equivalent to chronic bronchitis is a non-specific series of changes in the bronchial wall generally characterized by an increase in the size and number of mucous glands and an increased number of goblet cells in the epithelium. When progressing into a chronic condition, bronchitis is a serious and incurable disorder. Consequently, a composition according to the present invention may be effective in controlling a series of changes in the bronchial wall generally characterized by an increase in the size and number of mucous glands and an increase in the number of goblet cells. A composition according to the present invention may also be capable of reducing and/or eliminating any mucos production including an increased mucos production.

Bronchial infections usually remain confined to the mucosa, and some resolve spontaneously without the need for treatment. Chronic bronchitis affects both the large and small airways. In the large airways, hypertrophy and hyperplasia of glandular structures and goblet cell metaplasia are prominent features of the condition. In the small airways, peribronchiolar fibrosis and airway narrowing may be prominent features. In chronic bronchitis hypertrophy of glandular structures and goblet cell metaplasia in the proximal airways likely contribute to an increased mucus production, the expectoration of which is one defining characteristic of chronic bronchitis. Consequently, a composition according to the present invention may be effective in preventing an airflow limitation in a subject prone to contracting bronchitis and/or to alleviate any airflow limitation or obstruction already present in said subject. Particularly, a composition according to the present invention may be effective in controlling hypertrophy and hyperplasia of glandular structures and goblet cell metaplasia, as well as peribronchiolar fibrosis and a narrowing of the small airways.

Bronchitis may be caused by a number of factors including viral and/or bacterial infection, environmental pollutants including cigarette smoke, and allergy. These factors may occur together or separately. A viral infection may e.g. predispose an individual to a subsequent bacterial infection. Bronchial infections occur in patients with abnormal airways who have reduced host defenses. The three major bacterial pathogens isolated during bronchial infections are non-typable Haemophlus influenzae, moraxella catarrhalis, and Streptococcus pneumoniae. A composition according to the present invention may especially be effective in preventing viral and/or bacterial infection in a subject by e.g. increasing the host defenses of said subject.

The term small airways as used herein is defined as small bronchi and bronchioles that contain no cartilage, glands or alveoli in their walls and measure 2 mm or less in internal diameter. The term small airways disease is used for a group of non-specific histological changes of peripheral airways found in individuals with a limited or obstructed airflow, including individuals having features such as mucus plugging, chronic inflammation, and muscular enlargement of small airway walls. Small airways disease is present in some patients with the clinical picture of chronic bronchitis. Consequently, a composition according to the present invention may be effective in preventing, treating, prophylactically treating and/or alleviating a limited or obstructed flow of air through the small airways.

In small or peripheral airways disease, there is inflammation of bronchioli and mucous metaplasia and hyperplasia, increased intraluminal mucus and increased wall muscle. Consequently, a composition according to the present invention may be effective in controlling inflammation of the bronchioli and mucous metaplasia and hyperplasia, and effective in reducing and/or eliminating any increased intraluminal mucus formation and/or any increased wall muscle development.

### BRIEF DESCRIPTION OF THE DRAWINGS

FIG. 1 shows the levels of total cholesterol (mM) for the four groups of patients in Example 2 (A: Patients in Group I; B: Patients receiving placebo with the same intake of protein and fiber as Group I; C: Patients in Group I; D: Patients receiving placebo with the same intake of protein and fiber as Group II) at each of the 8 visits. TC=total cholesterol.

FIG. 2 shows the levels of LDL-cholesterol (mM) for the four groups of patients in Example 2 (A: Patients in Group I; B: Patients receiving placebo with the same intake of protein and fiber as Group I; C: Patients in Group I; D: Patients receiving placebo with the same intake of protein and fiber as Group II) at each of the 8 visits. LDL-chol=LDL-cholesterol.

FIG. 3 shows the levels of HDL-cholesterol (mM) for the four groups of patients in Example 2 (A: Patients in Group I; B: Patients receiving placebo with the same intake of protein and fiber as Group I; C: Patients in Group I; D: Patients receiving placebo with the same intake of protein and fiber as Group II) at each of the 8 visits. HDL-chol=HDL-cholesterol.

FIG. 4 shows the levels of triglycerides (mM) for the four groups of patients in Example 2 (A: Patients in Group I; B: Patients receiving placebo with the same intake of protein and fiber as Group I; C: Patients in Group I; D: Patients receiving placebo with the same intake of protein and fiber as Group II) at each of the 8 visits.

FIG. 5 shows the mean ratio of serum HDL/LDL-cholesterol for the four groups of patients in Example 2 (A: Patients in Group I; B: Patients receiving placebo with the same intake of protein and fiber as Group I; C: Patients in Group I; D: Patients receiving placebo with the same intake of protein and fiber as Group II) at each of the 8 visits.

### DETAILED DESCRIPTION OF THE INVENTION

A composition according to the present invention comprises a novel combination of soy protein, preferably isolated soy protein, a phytoestrogen compound, preferably naturally occurring isoflavones, and dietary fibers, preferably soybean fibers, more preferably soy cotyledon fibers.

The soy protein can be provided by isolated soy protein, soy protein concentrate, soy flour or the like or any combination thereof. Isolated soy protein is preferred.

Isolated soy protein is the major proteinacious fraction of soybeans. It is prepared from high quality, dehulled, defatted soybeans by removing a preponderance of the non-protein components resulting in an isolated soy protein fraction which in the present context shall contain at least 90 percent protein (N×6.25) on a moisture free basis. The preparation takes place through a series of steps in which the soybean protein portion is separated from the rest of the soybean. The removal of carbohydrate results in a product, which is essentially bland in flavor and therefore particularly useful in a nutritional composition for humans.

Soy protein concentrates are made by removing most of the oil and water-soluble non-protein constituents from defatted and dehulled soybeans. In the present context a soy protein concentrate shall preferably contain at least 65 percent protein on a moisture-free basis.

The soy protein can also be provided by soy flour, which can be full-fat or defatted soy flour. Full-fat soy flour comes from whole, dehulled soybeans that have been ground into a fine powder and, as the name implies, still contains the fat naturally found in soybeans. Defatted soy flour comes from whole, dehulled, defatted soybeans that have been ground into a fine powder. Soy flour is approximately 50 percent soy protein on a dry weight basis in the present context.

The soy protein used in a composition according to the present invention should preferably supply all the essential amino acids in the amounts required for humans. Preferably, the soy protein should also meet or exceed the essential amino acid requirement pattern for children and adults as established by the Food and Agricultural Organization, World Health Organization and United Nations University (FAO/WHO, UNU). Furthermore, the preferred soy protein should be comparable in digestibility to milk, meat, fish, and egg protein. Finally, the preferred soy protein shall be effective in maintaining nitrogen balance when consumed at the recommended protein intake level.

Preferred isolated soy protein products meeting the foregoing requirements are supplied by Protein Technologies International, Inc. under the brand name SUPRO®.

SUPRO® isolated soy proteins are supplied in many different qualities and SUPRO® XT 12C is one particularly preferred quality. The currently most preferred quality is termed SUPRO® FXP-HO159.

The soy protein is preferably the main or sole protein source in a nutritional composition according to the present invention. However, parts of the protein source may be provided by other proteins such as e.g. skimmed milk, preferably as a powder, and other vegetable or animal proteins including diary proteins. Preferably, at least 45 weight percent, such as 50 weight percent, for example at least 60 weight percent, such as at least 70 weight percent, for example at least 75 weight percent, such as at least 80 weight percent, for example at least 85 weight percent, such as at least 90 weight percent, for example at least 95 weight percent, such as at least 98 weight percent of the total protein content of the composition is soy protein, and more preferably substantially all of the protein is soy protein.

In a preferred embodiment of the invention the soy protein is provided by isolated soy protein. In this embodiment, preferably at least 50 weight percent, for example at least 60 weight percent, such as at least 70 weight percent, for example at least 75 weight percent, such as at least 80 weight percent, for example at least 85 weight percent, such as at least 90 weight percent, for example at least 95 weight percent, such as at least 98 weight percent of the total protein content of the composition is isolated soy protein, and more preferably substantially all of the protein is provided by isolated soy protein.

The total protein content of a composition according to the present invention provides at least 15 percent of the total energy content of the composition, for example 18 percent, such as at least 20 percent, for example at least 22 percent, such as at least 25 percent, for example at least 28 percent, such as at least 30 percent, for example at least 32 percent, such as at least 35 percent, for example at least 38 percent, such as at least 40 percent, for example at least 42 percent, such as at least 45 percent, for example at least 48 percent, such as at least 50 percent of the total energy content of the composition, and preferably less than 90 percent of the total energy content of the composition.

Phytoestrogen compounds according to the present invention are defined as naturally occurring plant substances, said substances being either structurally or functionally similar to 17β-estradiol or generating estrogenic effects. Phytoestrogens consist of a number of classes including isoflavones, coumestans, lignans and resorcylic acid lactones. Examples of isoflavones according to the present invention are genistein, daidzein, equol, glycitein, biochanin A, formononetin, and O-desmethylangolesin. The phytoestrogen compounds of a composition according to the present invention are preferably isoflavones, more preferably genistein, daidzein, glycitein and/or equol, yet more preferably genistein and/or daidzein, and even more preferably genistein. A preferred composition according to the present invention may accordingly comprise a single isoflavone, such as genistein, daidzein, glycitein or equol, or it may comprise at least one isoflavone selected from the group comprising at least genistein, daidzein, glycitein and equol. When present in the plant the isoflavones are mainly in a glucoside form, i.e. attached to a sugar molecule. This glucoside form can be deconjugated to yield a so-called aglycone form, which is the biologically active species. A composition according to the present invention may comprise isoflavones in glucoside and/or aglycone forms regardless of whether the deconjugation to the aglycone form has taken place biologically, in vitro or by any other means whereby the isoflavones are included in a composition according to the present invention or if the aglycone forms are the native form of the isoflavones.

The phytoestrogen compound is preferably present in an amount of at least about 0.12 weight percent of the soy protein content, such as at least about 0.14 weight percent, for example at least about 0.16 weight percent, such as at least about 0.18 weight percent, for example at least about 0.20 weight percent, such as at least about 0.22 weight percent, for example at least about 0.24 weight percent, such as at least about 0.25 weight percent, for example more than about 0.25 weight percent, such as at least about 0.26 weight percent, for example at least about 0.28 weight percent, such as at least about 0.30 weight percent, for example at least about 0.32 weight percent, such as at least about 0.33 weight percent, for example more than about 0.33 weight percent, such as at least about 0.35 weight percent, for example at least about 0.40 weight percent, such as at least about 0.45 weight percent, for example at least about 0.50 weight percent, such as at least about 0.55 weight percent, for example at least about 0.60 weight percent, such as at least about 0.65 weight percent, for example at least about 0.70 weight percent, such as at least about 0.75 weight percent, for example at least about 0.80 weight percent, such as at least about 0.85 weight percent, for example at least about 0.90 weight percent, such as at least about 1.0 weight percent of the soy protein content, and preferably less than 2.50 weight percent of the soy protein content.

In the past, the downstream processing techniques used in the preparation of soy proteins have included steps that removed and/or destroyed isoflavones. Methods are available today, which provide soy protein products with high, fixed levels of naturally occurring isoflavones. The isoflavones according to the present invention in glucoside and/or aglycone forms can be included in a composition according to the present invention as part of such soy protein products and/or by themselves and/or as part of any other composition comprising isoflavones.

The dietary fibers used in the present invention should preferably comprise a mixture of insoluble fibers and water-soluble fibers also referred to as soluble fibers. Soluble fibers have a lowering effect on-blood cholesterol levels. Examples of dietary fibers comprising soluble fibers are fibers from apples, bananas, oranges, carrots, oats, and soybeans. The dietary fibers preferably comprise soluble fibers in an amount of about 5 weight percent, such as about 10 weight percent, for example about 15 weight percent, such as about 20 weight percent, for example about 25 weight percent, such as about 30 weight percent, for example about 35 weight percent, such as about 40 weight percent, for example about 45 weight percent, such as about 50 weight percent, for example about 55 weight percent, such as about 60 weight percent, for example about 65 weight percent, such as about 70 weight percent, for example about 75 weight percent, such as about 80 weight percent, for example about 85 weight percent, such as about 90 weight percent, for example about 95 weight percent. The dietary fibers used in the present invention are preferably soybean fibers, more preferably soy cotyledon fibers. Such fibers are derived from dehulled and defatted soybean cotyledon and are comprised of a mixture of soluble and insoluble fibers. Soy cotyledon fibers are distinctly different from soybean fibers derived from soy hulls as well as other fiber sources. Soy cotyledon fibers are bland tasting, contain no cholesterol, are low in fat and sodium, and they have good water-binding properties and low caloric content.

Soy cotyledon fibers supplied in a fat-modified and low-cholesterol diet are known to further reduce serum cholesterol levels in a subject suffering from mild to severe hypercholesterolemia. The effect is a lowering of the serum levels of total cholesterol including a lowering of the serum levels of LDL-cholesterol. However, HDL-cholesterol and total triglycerides are not significantly affected by soy cotyledon fibers. Soybean fibers, in particular soy cotyledon fibers, are believed to provide a synergistic effect in combination with soy protein and/or with a phytoestrogen compound, such as naturally occurring isoflavones, or to exert a potentiating effect on the soy protein and/or the phytoestrogen compound, said synergistic or potentiating effect being effective in lowering serum levels of lipid and cholesterol in subjects having normal as well as elevated serum levels of total cholesterol and total triglycerides.

Without wishing to be bound by any specific theory it is presently believed that both soluble dietary fibers (working as nutrients) and insoluble dietary fibers (working as bulking agents), in particular from soybean fibers, more particularly soy cotyledon fibers, provide favorable growth conditions for the microflora in the human gut, which makes the microflora more effective in deconjugating isoflavones in the glucoside form to the aglycone form. Isoflavones in the aglycone form are absorbed faster and to a greater extent in the human gut than isoflavones in the glucoside form, and isoflavones in the aglycone form are the biologically more active species in the present context. In view hereof it can be understood that administration of a combination of soy proteins, a high, fixed level of isoflavones and a combination of soluble and insoluble fibers may be effective in providing an increased uptake of isoflavones.

The amount of dietary fibers of the total weight of a composition according to the present invention on a dry basis is preferably more than 4 weight percent, for example at least 5 weight percent, such as at least 6 weight percent, for example at least 7 weight percent, such as at least 8 weight percent, for example at least 9 weight percent, such as at least 10 weight percent, for example at least 11 weight percent, such as at least 12 weight percent, for example at least 13 weight percent, such as at least 14 weight percent, for example at least 15 weight percent, such as at least 16 weight percent, for example at least 17 weight percent, such as at least 18 weight percent, for example at least 19 weight percent, such as at least 20 weight percent, and preferably less than 50 weight percent.

Preferred amounts of dietary fibers as a weight percent of the content of soy protein, shall be in the range of from about 10 to 100 weight percent, and preferred amounts are in the range of from 15 to 90 weight percent, such as from 20 to 80 weight percent, for example 25 weight percent, such as 30 weight percent, for example 33 weight percent, such as 35 weight percent, for example 40 weight percent, such as 50 weight percent, for example 60 weight percent, such as 70 weight percent, for example 75 weight percent.

Accordingly, the weight ratio of soy protein to dietary fibers is from about 1.0 to about 10.0, preferably more than about 1.0, for example about 1.25, such as at least about 1.5, for example at least about 1.75, such as at least about 2.0, for example at least about 2.25, such as at least about 2.5, for example at least about 2.75, such as at least about 3.0, for example at least about 3.25, such as at least about 3.5, for example at least about 3.75, such as at least about 4.0, for example at least about 4.25, such as at least about 4.5, for example at least about 4.75, such as at least about 5.0, for example at least about 5.5, such as at least about 6.0, for example at least about 7.5.

The preferred daily dosage of soybean fibers is from at least 1 g to about 100 g soybean fibers, for example from at least 2 to about 75 g soybean fibers, such as from at least 3 g to about 50 g, for example from at least 4 g to about 40 g, such as from at least 5 to about 30 g, such as from at least 10 g to about 20 g soybean fibers.

Preferred soy cotyledon fiber products manufactured by Protein Technologies International, Inc. are marketed under the brand name of FIBRIM®. Among the various soybean fibers produced under the FIBRIM® brand, FIBRIM® 1020 is particularly preferred because of a particularly pleasant mouth feel and dispersability for dry blended beverage applications. FIBRIM® 2000 is presently preferred for use in ready-made liquids.

Two compositions of isolated soy protein and soy cotyledon fiber are preferred in order to maximize the content of soy protein and isoflavones contained therein namely SUPRO® FXP-HO159 and FIBRIM® 1020 for dry blended beverage applications and SUPRO® FXP-HO159 and FIBRIM® 2000 for use in ready made liquids.

Alternatively, the present invention provides a composition wherein no soy protein is present and wherein the dietary fibers are soy cotyledon fibers. This composition comprises soy cotyledon fibers in an amount of more than 4 weight percent of the total weight of the composition on a dry basis and at least one phytoestrogen compound in an amount of more than 0.10 weight percent of the soy cotyledon fibers of the composition. The present invention also provides the use of such a composition as a medicament and/or in the manufacture of a medicament effective in preventing, treating, prophylactically treating, alleviating and/or eliminating a cardiovascular disease in a subject. The present invention also provides the use of such a composition as a medicament and/or in the manufacture of a medicament effective in preventing, treating, prophylactically treating, alleviating and/or eliminating arteriosclerosis or a related cardiovascular disease in a subject. The present invention also provides the use of such a composition as a medicament and/or in the manufacture of a medicament effective in treating and/or alleviating type 2 diabetes, the metabolic syndrome and/or cardiovascular diseases associated therewith in a subject. The present invention also provides the use of such a composition as a medicament and/or in the manufacture of a medicament effective in treating and/or alleviating a pulmonary disease in a subject, said treatment and/or alleviation resulting in an increased FEV₁ of a subject, as measured by forced expiratory volume in the first second of expiration. The present invention also provides the use of such a composition and/or such a composition for use in treating arteriosclerosis or a related cardiovascular disease in a subject. The present invention also provides the use of such a composition and/or such a composition for use in treating and/or alleviating type 2 diabetes, the metabolic syndrome and/or cardiovascular diseases associated therewith in a subject. The present invention also provides the use of such a composition and/or such a composition for use in treating pulmonary diseases in a subject.

When no soy protein is present in the composition, the phytoestrogen compound is preferably present in an amount of at least about 0.12 weight percent of the soy cotyledon fiber content, such as at least about 0.14 weight percent, for example at least about 0.16 weight percent, such as at least about 0.18 weight percent, for example at least about 0.20 weight percent, such as at least about 0.22 weight percent, for example at least about 0.24 weight percent, such as at least about 0.25 weight percent, for example more than about 0.25 weight percent, such as at least about 0.26 weight percent, for example at least about 0.28 weight percent, such as at least about 0.30 weight percent, for example at least about 0.32 weight percent, such as at least about 0.33 weight percent, for example more than about 0.33 weight percent, such as at least about 0.35 weight percent, for example at least about 0.40 weight percent, such as at least about 0.45 weight percent, for example at least about 0.50 weight percent, such as at least about 0.55 weight percent, for example at least about 0.60 weight percent, such as at least about 0.65 weight percent, for example at least about 0.70 weight percent, such as at least about 0.75 weight percent, for example at least about 0.80 weight percent, such as at least about 0.85 weight percent, for example at least about 0.90 weight percent, such as at least about 1.00 weight percent, for example at least about 1.25 weight percent, such as at least about 1.50 weight percent, for example at least about 1.75 weight percent, such as at least about 2.00 weight percent, for example at least about 2.50 weight percent, such as at least about 3.00 weight percent, for example at least about 3.5 weight percent, such as at least about 5.00 weight percent of the soy cotyledon fiber content of the composition, and preferably less than 10.00 weight percent of the soy cotyledon fiber content of the composition.

Alternatively, the present invention provides a composition wherein no dietary fibers are present. This composition comprises soy protein, preferably isolated soy protein in an amount of at least 50 weight percent of the total protein content of the composition, said total protein content providing at least 15 percent of the total energy content of the composition, and at least one phytoestrogen compound in an amount of more than 0.10 weight percent of the soy protein content of the composition. The present invention also provides the use of such a composition as a medicament and/or in the manufacture of a medicament effective in preventing, treating, prophylactically treating, alleviating and/or eliminating a cardiovascular disease in a subject. The present invention also provides the use of such a composition as a medicament and/or in the manufacture of a medicament effective in preventing, treating, prophylactically treating, alleviating and/or eliminating arteriosclerosis or a related cardiovascular disease in a subject. The present invention also provides the use of such a composition as a medicament and/or in the manufacture of a medicament for treating diabetic subjects, said treatment being effective in lowering serum levels of glucose and/or insulin and/or lipids. The present invention also provides the use of such a composition as a medicament and/or in the manufacture of a medicament effective in treating and/or alleviating type 2 diabetes, the metabolic syndrome as defined herein and/or cardiovascular diseases associated therewith in a subject. The present invention also provides the use of such a composition as a medicament and/or in the manufacture of a medicament effective in treating subjects diagnosed as having a pulmonary disease, said treatment being effective at least in increasing FEV₁ of a subject, as measured by forced expiratory volume in the first second of expiration. The present invention also provides the use of such a composition as a medicament and/or in the manufacture of a medicament effective in treating and/or alleviating pulmonary diseases in a subject. The present invention also provides the use of such a composition and/or such a composition for use in treating arteriosclerosis or a related cardiovascular disease in a subject. The present invention also provides the use of such a composition and/or such a composition for use in the treatment of diabetic subjects, said treatment being particularly effective in lowering serum levels of glucose and lipids in a subject. The present invention also provides the use of such a composition and/or such a composition for use in the treatment and/or alleviation of a pulmonary disease in a subject, said treatment and/or alleviation resulting in an increased FEV₁ of a subject, as measured by forced expiratory volume in the first second of expiration.

A composition according to the present invention may optionally comprise a carbohydrate source, a fat source, flavoring agents, vitamins, minerals, electrolytes, trace elements and other conventional additives. The nutritional composition according to the present invention may in one embodiment also comprise one or more flavoring agents such as cocoa, vanilla, lime, strawberry or soup flavors, such as mushroom, tomato or bouillon and/or sweeteners such as aspartame as well as other additives such as xanthan gum.

When a carbohydrate source is present in a composition according to the present invention, it is preferably present in an amount of less than 30 weight percent such as less than 25 weight percent of the composition. Preferably, the amount of carbohydrate amounts to at least 5 weight percent, more preferred at least 10 weight percent, and most preferred at least 15 weight percent, of the composition. The preferred carbohydrates for use in a composition according to the present invention are dextrose, fructose and/or maltodextrin, or glucose. Skimmed milk and lecithinated fat reduced cacao are other possible carbohydrate sources. When a composition according to the present invention is for use in the prevention and/or treatment of type 2 diabetes, the metabolic syndrome and associated cardiovascular diseases, lecithinated fat reduced cacao is particularly preferred. Other preferred carbohydrates for use in a composition according to the present invention for use in the prevention and/or treatment of type 2 diabetes, the metabolic syndrome and associated cardiovascular diseases are polydextrose or saccharose, but these should be limited using other sweeteners like e.g. aspartame.

When a fat source is present in a composition according to the present invention, it is usually present in an amount from 0.5 to 10 weight percent, preferably 1 to 9 weight percent, such as from 1.5 to 8 weight percent, for example from 2 to 7 weight percent, such as from 2.5 to 6 weight percent of the composition. The fat source will preferably comprise polyunsaturated fatty acids and monounsaturated fatty acids and optionally also saturated fatty acids. Soy lecithins and α-linolenic acid are particularly preferred. The fat source will preferably comprise polyunsaturated fatty acids and monounsaturated fatty acids and optionally also saturated fatty acids. The amount of polyunsaturated fatty acids and monounsaturated fatty acids, including the essential fatty acids, may range from 35 to 50, preferably 38 to 44, weight percent of the total amount of the fat source. The essential fatty acids are also called omega-6 and omega-3 fatty acids and include linolic acid and/or linolenic acid (α-linolenic acid). The amount of saturated fatty acids may be from 20 to 30 weight percent, preferably 22 to 26 weight percent, of the total amount of fat.

Vitamins and minerals may optionally be added to a composition according to the present invention in accordance with the limits laid down by health authorities. A composition according to the present invention may comprise all recommended vitamins and minerals. The vitamins will typically include A, B1, B2, B12, folic acid, niacin, panthotenic acid, biotin, C, D, E and K. The minerals will typically include iron, zinc, iodine, copper, manganese, chromium and selenium. Electrolytes, such as sodium, potassium and chlorides, trace elements and other conventional additives may also be added in recommended amounts.

A preferred composition can be obtained by mixing:

| | Content per | Grams per |
|---|---|---|
| | 100 gram (%) | serving |
| Isolated soy protein (SUPRO ® | 50.00 | 18.5 |
| FXP-HO159) | | |
| Soybean fibers (FIBRIM ® 1020) | 12.50 | 4.63 |
| Fructose | 22.62 | 8.37 |
| Lecithinated fat reduced cocoa | 9.30 | 3.44 |
| Soy lecithin | 3.55 | 1.31 |
| Flavorings | 1.28 | 0.47 |
| Xanthan gum | 0.50 | 0.19 |
| Aspartame | 0.25 | 0.09 |

The above-mentioned composition in an amount of preferably about 37 grams corresponds to one serving of a daily diet. The composition has an energy content of about 339 kcal (1,437 kJ) per 100 grams.

Another preferred composition can be obtained by mixing

| | Content per |
|---|---|
| | 100 gram (%) |
| Isolated soy protein (SUPRO ® FXP-HO159) | 50.00 |
| Soybean fibers (FIBRIM ® 1020) | 16.70 |
| Carbohydrates | 18.20 |
| Lecithinated fat reduced cocoa | 9.30 |
| Soy lecithin | 3.55 |
| Flavorings | 1.25 |
| Xanthan gum | 0.50 |
| Aspartame | 0.50 |

A composition according to the present invention may be used for special dietary use, preferably for lowering serum levels of total cholesterol and/or LDL-cholesterol and/or triglycerides in subjects such as hyperlipidemic patients or normocholesterolemic patients suffering from a cardiovascular disease, and/or for lowering serum levels of glucose and/or insulin and/or total cholesterol and/or LDL-cholesterol and/or triglycerides and/or for increasing glucose tolerance and/or insulin sensitivity and/or for preventing, treating and/or alleviating impaired glucose tolerance and/or insulin secretory failure in diabetic subjects and/or for preventing, treating and/or alleviating an arteriosclerotic condition by reducing the influx of lipoproteins and/or cholesterol and/or triglycerides into the endocelium of the arterial wall of a diabetic subject suffering from a cardiovascular disease. For example, from one to three daily meals of ordinary food can be supplemented or replaced by a composition according to the present invention. Hereby, significant reductions in serum levels of cholesterol and/or LDL-cholesterol and/or triglycerides can be obtained, as well as an improvement of serum HDL/LDL-cholesterol ratio and/or an increase in serum HDL-cholesterol levels. The composition may provide from about 50 to about 250 kcal per serving.

The daily dose of a composition according to the present invention may comprise an energy content of from 400 to 800, in particular from 450 to 800 kcal/day, which is considered to be a very low calorie diet (VLCD), or it may comprise an energy content of from 800 to 1200 kcal/day, which is considered to be a low-calorie diet (LCD). In another medical embodiment of the present invention, the energy content may correspond to the daily energy requirement of a normal person.

The present invention also provides a composition according to the invention in the form of a micronutrient. In this connection a micronutrient is a nutritional supplement and/or a pharmacological composition and/or a medicament comprising i) a synthetic phytoestrogen-like compound capable of binding to an estrogen receptor or an estrogen-like receptor, and/or ii) a naturally occurring, plant-extractable compound in an amount, on a weight per weight basis, in excess of the amount of said compound, when it is present in a natural host such as a plant cell from which the compound can be extracted or isolated, and optionally iii) soy peptides obtainable from a partial hydrolysis of soy protein.

The naturally occurring, plant-extractable compound is preferably but not limited to compounds capable of binding to an estrogen receptor, an estrogen-like receptor, a beta-2-adrenergic receptor or a receptor belonging to the class of beta-2-adrenergic receptors. When the naturally occurring compounds are isolated from plants such as soybeans, they may be selected from the group at least containing phytoestrogens such as soybean phytoestrogens such as soybean isoflavones, soy protein or fragments thereof, e.g. peptides or amino acid sequences, soybean fibers, lecithin, linolenic acid, an antioxidant, a saponin, a lignan, a protease inhibitor, a trypsin inhibitor, and a tyrosine kinase inhibitor. Additional constituents of the micronutrient may preferably be selected among a DNA topoisomerase inhibitor, a ribosome kinase inhibitor, a growth control factor such as e.g. epidermal growth factor, transforming growth factor alpha, platelet derived growth factor, and preferably any growth control factor controllable by a tyrosine kinase activity. The micronutrient may also comprise ormeloxifene and/or levormeloxifene as described by among others Holm et al. (1997) in Arteriosclerosis, Thrombosis, and Vascular Biology 17 (10), 2264-2272, and in Clinical Investigation, 100 (4), 821-828. When the naturally occurring compound is an isoflavone, the isoflavone may have been deconjugated to the aglycone form either biologically or in vitro prior to the incorporation in the micronutrient.

In one particularly preferred embodiment the present invention provides a composition or a micronutrient according to the present invention in combination with a functional food ingredient comprising a sterol, preferably an ingredient selected from the group comprising a stanol ester, a tocotrienol, a mevinolin, and a phytosterol compound such as e.g. campesterol, sitosterol or stigmasterol, or a combination thereof.

According to one preferred embodiment, a composition or a micronutrient according to the present invention is for use as a functional food ingredient. A composition or a micronutrient according to the present invention may also be administered as a probe or by intravenous administration, or in tablet or capsule form. The present invention also provides a pharmaceutical preparation comprising the a composition or a micronutrient according to the present invention, use of the a composition or a micronutrient according to the present invention in therapy and/or a diagnostic method performed on the human or animal body, use of a composition or a micronutrient according to the present invention in the manufacture of a medicament, use of a composition or a micronutrient according to the present invention in the manufacture of a medicament for treating a subject suffering from cardiovascular diseases, use of a composition or a micronutrient according to the present invention in the manufacture of a medicament for treating a subject suffering from type 2 diabetes, the metabolic syndrome and/or cardiovascular diseases associated therewith in a diabetic subject and use of a composition or a micronutrient according to the present invention in the manufacture of a medicament for treating a subject suffering from pulmonary diseases.

The micronutrient is particularly useful in preventing, treating, prophylactically treating and/or alleviating hypercholesterolemia, hypertriglyceridemia, other hyperlipidemias, arteriosclerosis, atherosclerosis and/or related cardiovascular diseases and in preventing and/or treating type 2 diabetes, the metabolic syndrome and/or cardiovascular diseases associated therewith in a diabetic subject and in preventing, treating, prophylactically treating and/or alleviating a pulmonary disease such as e.g. a disease selected from the group comprising inflammation of the airways, bronchoconstriction, bronchitis, asthma, and small airways diseases.

In one embodiment the present invention provides a composition according to the present invention for use as a medicament or as a dietary preparation. A composition according to the present invention for use as a medicament or as a dietary preparation may preferably be used in preventing, treating, prophylactically treating and/or alleviating cardiovascular diseases such as e.g. a disease selected from the group comprising hypercholesterolemia, hypertriglyceridemia, other hyperlipidemias, arteriosclerosis, atherosclerosis, arteriolosclerosis, coronary heart disease, angina pectoris, thrombosis, myocardial infarction, and hypertension, in a subject, preferably for use in preventing, treating, prophylactically treating and/or alleviating arteriosclerosis and/or atherosclerosis in a subject . A composition according to the present invention for use as a medicament or as a dietary preparation may also preferably be used in preventing, treating, alleviating and/or eliminating type 2 diabetes. A composition according to the present invention for use as a medicament or as a dietary preparation may also preferably be used in preventing, treating, alleviating and/or eliminating a cardiovascular disease, such as e.g. hypercholesterolemia, hypertriglyceridemia, hypertension, hyperglycemia, hyperinsulinemia, arteriosclerosis, atherosclerosis, arteriolosclerosis, coronary heart disease, angina pectoris, thrombosis, and myocardial infarction, in a diabetic subject. A composition according to the present invention for use as a medicament or as a dietary preparation may also preferably be used for preventing and/or treating pulmonary diseases, such as preferably a disease selected from the group comprising inflammation of the airways, bronchoconstriction, bronchitis, asthma, and small airways diseases, in a subject.

The present invention also provides the use of a composition according to the present invention as a medicament and/or in the manufacture of a medicament for preventing, treating, prophylactically treating and/or alleviating cardiovascular diseases such as e.g. a disease selected from the group comprising hypercholesterolemia, hypertriglyceridemia, other hyperlipidemias, arteriosclerosis, atherosclerosis, arteriolosclerosis, coronary heart disease, angina pectoris,- thrombosis, myocardial infarction, and hypertension, particularly a disease selected from the group comprising arteriosclerosis and atherosclerosis, in a subject. The present invention also provides the use of a composition according to the present invention as a medicament and/or in the manufacture of a medicament for preventing, treating and/or alleviating type 2 diabetes and/or the metabolic syndrome in a subject and/or a cardiovascular disease in a diabetic subject. The present invention also provides the use of a composition according to the present invention as a medicament and/or in the manufacture of a medicament for preventing, treating, prophylactically treating and/or alleviating pulmonary diseases such as e.g. a disease selected from the group comprising inflammation of the airways, bronchoconstriction, bronchitis, asthma, and small airways diseases, in a subject.

The composition according to the present invention is effective in lowering levels of cholesterol in normocholesterolemic patients by at least 2%, for example at least 5%, such as at least 8%, for example at least 10%, such as at least 12%, for example at least 14%, such as at least 16%, for example at least 18%, such as at least 20%, for example at least 25%, such as at least 30%. The composition according to the present invention is effective in lowering levels of triglycerides in normocholesterolemic patients by at least 10%, such as at least 12%, for example at least 14%, such as at least 16%, for example at least 18%, such as at least 20%, for example at least 25%, such as at least 30%.

The composition according to the present invention is effective in lowering levels of cholesterol in mildly hypercholesterolemic patients by at least 3%, for example at least 5%, such as at least 8%, for example at least 10%, such as at least 12%, for example at least 15%, such as at least 20%, for example at least 25%, such as at least 30%, for example at least 35%, such as at least 40%, for example at least 45%. The composition according to the present invention is effective in lowering levels of triglycerides in mildly hypercholesterolemic patients by at least 15%, such as at least 20%, for example at least 25%, such as at least 30%, for example at least 35%, such as at least 40%, for example at least 45%.

The composition according to the present invention is effective in lowering levels of cholesterol in severely hypercholesterolemic patients by at least 3%, for example at least 5%, such as at least 8%, for example at least 10%, such as at least 12%, for example at least 15%, such as at least 20%, for example at least 25%, such as at least 30%, for example at least 35%, such as at least 40%, for example at least 45%, such as at least 50%, for example at least 55%, such as at least 60%. The composition according to the present invention is effective in lowering levels of triglycerides in severely hypercholesterolemic patients by at least 20%, for example at least 25%, such as at least 30%, for example at least 35%, such as at least 40%, for example at least 45%, such as at least 50%, for example at least 55%, such as at least 60%.

A composition according to the present invention for use as a medicament and/or the use of a composition according to the present invention as a medicament and/or in the manufacture of a medicament for preventing, treating, prophylactically treating and/or alleviating cardiovascular diseases in a subject may be effective in reducing the influx of cholesterol and/or triglycerides into the arterial wall and/or reducing the amount of oxidized LDL-cholesterol present in the arterial wall and/or lowering serum levels of total cholesterol and/or LDL-cholesterol and/or homocystein and/or triglycerides and/or increasing the serum HDL/LDL-cholesterol ratio and/or increasing serum levels of HDL-cholesterol in a subject and/or preventing, reducing or eliminating fatty streak formation and/or preventing, reducing or eliminating fibrous plaque formation and/or preventing, reducing or eliminating complicated lesion formation and/or reducing or eliminating the risk of a subject contracting angina pectoris and/or reducing or eliminating the risk of a subject contracting a myocardial infarction.

A composition according to the present invention for use as a medicament and/or the use of a composition according to the present invention as a medicament and/or in the manufacture of a medicament for preventing and/or treating diabetes and/or the metabolic syndrome and/or a cardiovascular disease associated therewith in a subject may be effective in i) lowering serum glucose levels and/or ii) reducing the influx of cholesterol and/or triglycerides into the arterial wall and/or reducing the amount of oxidized LDL-cholesterol present in the arterial wall and/or iii) lowering serum levels of total cholesterol and/or LDL-cholesterol and/or triglycerides and/or homocystein and/or increasing the serum HDL/LDL-cholesterol ratio and/or serum HDL-cholesterol levels and/or iv) increasing glucose tolerance and/or insulin sensitivity and/or v) alleviating impaired glucose tolerance and/or insulin secretory failure and/or improving insulin secretion and/or vi) preventing, treating, alleviating,and/or eliminating cardiovascular diseases, such as e.g. hypercholesterolemia, hypertriglyceridemia, other hyperlipidemias, arteriosclerosis, atherosclerosis, arteriolosclerosis, coronary heart disease, angina pectoris, thrombosis, myocardial infarction, hypertension, hyperglycemia, and hyperinsulinemia, in a diabetic subject and/or preventing, reducing or eliminating fatty streak formation and/or preventing, reducing or eliminating fibrous plaque formation and/or preventing, reducing or eliminating complicated lesion formation and/or reducing or eliminating the risk of a diabetic subject contracting angina pectoris and/or reducing or eliminating the risk of a diabetic subject contracting a myocardial infarction and/or in treating a procoagulant state and/or an increased activity of clotting factors, insulin resistance, glycosidation and/or oxidation and/or chemical modification of lipoproteins, as well as impaired glucose tolerance.

A composition according to the present invention for use as a medicament and/or the use of a composition according to the present invention as a medicament and/or in the manufacture of a medicament for preventing, treating, prophylactically treating and/or alleviating pulmonary diseases may be effective in i) preventing, treating, prophylactically treating and/or alleviating asthma and/or ii) reducing and/or eliminating mucus hypersecretion and/or dyspnea in a subject suffering from asthma and/or iii) increasing FEV₁ of a subject as measured by forced expiratory volume in the first second of expiration and/or iv) preventing, treating, prophylactically treating, alleviating and/or reducing inflammation of the airways and/or v) preventing, treating, prophylactically treating and/or alleviating bronchoconstriction.

In another embodiment the present invention provides the use of a composition according to the present invention in the treatment of cardiovascular diseases in the human or animal body in an amount effective in lowering serum levels of total cholesterol and/or LDL-cholesterol and/or triglycerides and/or homocystein and/or increasing the serum HDL/LDL-cholesterol ratio and/or serum HDL-cholesterol levels and/or reducing the influx of cholesterol and/or triglycerides into the arterial wall and/or reducing the amount of oxidized LDL-cholesterol present in the arterial wall and/or preventing, reducing or eliminating fatty streak formation and/or preventing, reducing or eliminating fibrous plaque formation and/or preventing, reducing or eliminating complicated lesion formation and/or reducing or eliminating the risk of a subject contracting angina pectoris and/or reducing or eliminating the risk of a subject contracting a myocardial infarction, and/or alleviating the clinical condition of patients contracting a myocardial infection. The cardiovascular disease is preferably a cardiovascular disease selected from the group comprising hypercholesterolemia, hypertriglyceridemia, other hyperlipidemias, arteriosclerosis, atherosclerosis, arteriolosclerosis, coronary heart disease, angina pectoris, thrombosis, myocardial infarction, and hypertension and more preferred selected from arteriosclerosis and atherosclerosis.

In another embodiment the present invention provides the use of a composition according to the present invention in the treatment of type 2 diabetes and/or the metabolic syndrome in an amount effective in lowering serum levels of glucose and/or total cholesterol and/or LDL-cholesterol and/or triglycerides and/or homocystein and/or reducing the influx of cholesterol and/or triglycerides into the arterial wall and/or reducing the amount of oxidized LDL-cholesterol present in the arterial wall and/or improving glucose tolerance and/or increasing insulin sensitivity and/or alleviating impaired glucose tolerance and/or improving insulin secretion and/or reducing or eliminating fatty streak formation and/or preventing, reducing or eliminating fibrous plaque formation and/or preventing, reducing or eliminating complicated lesion formation and/or preventing, reducing or eliminating the risk of a subject contracting angina pectoris and/or preventing, reducing or eliminating the risk of a subject contracting a myocardial infarction and/or preventing, treating, prophylactically treating, alleviating and/or eliminating hypertension and/or hyperglycemia and/or hyperinsulinemia and/or hypercholesterolemia and/or hypertriglyceridemia and/or arteriosclerosis and/or atherosclerosis and/or arteriolosclerosis in a diabetic subject.

In another embodiment the present invention provides the use of a composition according to the present invention in the treatment of a pulmonary disease in a human or animal body, preferably a disease selected from the group comprising inflammation of the airways, bronchoconstriction, bronchitis, asthma, and small airways diseases, in an amount effective in preventing, treating, prophylactically treating and/or alleviating inflammation of the airways and/or bronchoconstriction and/or bronchitis and/or small airways diseases and/or asthma and/or reducing and/or eliminating mucus hypersecretion and/or dyspnea in a subject suffering from asthma and/or increasing FEV₁ of a subject as measured by forced expiratory volume in the first second of expiration.

The present invention also provides a method of preventing, treating, prophylactically treating and/or alleviating by therapy a cardiovascular disease in the human or animal body such as an arteriosclerotic condition of a human or animal body, said method comprising administration of a composition according to the present invention in an amount effective in lowering serum levels of total cholesterol and/or LDL-cholesterol and/or triglycerides and/or homocystein and/or increasing the serum HDL/LDL-cholesterol ratio and/or serum HDL-cholesterol levels and/or reducing the influx of cholesterol and/or triglycerides into the arterial wall and/or reducing the amount of oxidized LDL-cholesterol present in the arterial wall and/or preventing, reducing or eliminating fatty streak formation and/or preventing, reducing or eliminating fibrous plaque formation and/or preventing, reducing or eliminating complicated lesion formation and/or reducing or eliminating the risk of a subject contracting angina pectoris and/or reducing or eliminating the risk of a subject contracting a myocardial infarction, and/or alleviating the clinical condition of patients contracting a myocardial infection. The cardiovascular disease is preferably a cardiovascular disease selected from the group comprising hypercholesterolemia, hypertriglyceridemia, other hyperlipidemias, arteriosclerosis, atherosclerosis, arteriolosclerosis, coronary heart disease, angina pectoris, thrombosis, myocardial infarction, and hypertension and more preferred selected from arteriosclerosis and atherosclerosis.

The present invention also provides a method of preventing and/or treating by therapy type 2 diabetes and/or the metabolic syndrome in a human or animal body, said method comprising administration to said human or animal body of a composition according to the present invention in an amount effective in lowering serum levels of glucose and/or total cholesterol and/or LDL-cholesterol and/or triglycerides and/or homocystein and/or reducing the influx of cholesterol and/or triglycerides into the arterial wall and/or reducing the amount of oxidized LDL-cholesterol present in the arterial wall and/or improving glucose tolerance and/or increasing insulin sensitivity and/or alleviating impaired glucose tolerance and/or improving insulin secretion and/or reducing or eliminating fatty streak formation and/or preventing, reducing or eliminating fibrous plaque formation and/or preventing, reducing or eliminating complicated lesion formation and/or preventing, reducing or eliminating the risk of a subject contracting angina pectoris and/or preventing, reducing or eliminating the risk of a subject contracting a myocardial infarction and/or preventing, treating, prophylactically treating, alleviating and/or eliminating hypertension and/or hyperglycemia and/or hyperinsulinemia and/or hypercholesterolemia and/or hypertriglyceridemia and/or arteriosclerosis and/or atherosclerosis and/or arteriolosclerosis in a diabetic subject.

The present invention also provides a method of preventing, treating, prophylactically treating and/or alleviating by therapy a pulmonary disease in a human or animal body, preferably a disease selected from the group comprising inflammation of the airways, bronchoconstriction, bronchitis, asthma, and small airways diseases, said method comprising administration to said human or animal body of a composition according to the present invention in an amount effective in preventing, treating, prophylactically treating and/or alleviating inflammation of the airways and/or bronchoconstriction and/or bronchitis and/or asthma and/or small airways diseases and/or reducing and/or eliminating mucus hypersecretion and/or dyspnea in a subject suffering from asthma and/or increasing FEV₁ of a subject as measured by forced expiratory volume in the first second of expiration.

The period of treatment is preferably in the range of from 1 to 12 months or more, such as from 2 weeks to 9 months, for example from 3 weeks to 6 months, such as from 4 weeks to 4 months, such as from 6 weeks to 3 months. However, the period of treatment shall not be limited to these periods and may e.g. be longer than 12 months, such as e.g. a lifelong treatment in order to prevent cardiovascular diseases or in order to prevent and/or alleviate type 2 diabetes and/or a cardiovascular disease in connection therewith or in order to, prevent pulmonary diseases.

In one embodiment the present invention provides a pharmaceutical preparation comprising a composition according to the present invention. The pharmaceutical preparation can be prepared in any way known to the skilled person.

In another embodiment the present invention provides the use of a composition according to the present invention as a nutritional preparation and/or in the manufacture of a nutritional preparation for lowering serum levels of glucose and/or total cholesterol and/or LDL-cholesterol and/or triglycerides and/or homocystein and/or increasing the serum HDL/LDL-cholesterol ratio and/or serum levels of HDL-cholesterol in a subject, including a diabetic subject, and/or for alleviating a pulmonary condition such as e.g. asthma. The nutritional preparation may take any form, which is suitable for human or animal consumption. In one preferred embodiment, the composition is a powdery mixture, which is suspendable, dispersible or emulsifiable in a liquid for human or animal consumption. The liquid is preferably a water-containing liquid such as e.g. water, coffee, tea or juice, including fruit juice. For such a purpose, the composition may be packed in a package intended for covering part of or the total nutritional requirement for a defined period of time, such as a period of e.g. three days or a week. The present invention also provides the nutritional preparation in the form of a dietary supplement.

The nutritional preparation in one embodiment of the present invention is preferably a functional food or drink, i.e. a readily obtainable edible or drinkable substance that is supplemented with a composition according to the present invention to provide a medical or pharmaceutical effect. Accordingly, the present invention provides a composition according to the present invention for use as a functional food ingredient. Functional foods and drinks are preferably selected from the group comprising diary products, such as yogurt and yogurt ice cream, juice, such as orange juice or tomato juice, ready made liquids for drinking, a spreadable product such as e.g. a margarine or a vegetable or plant extracted oil, a cereal product, such as a traditional breakfast cereal product, nutritional bars, biscuits, bread, soups, such as tomato soup, a meat product, such as a hamburger, a meat substitute product, and a vegetable product. In a further embodiment, a nutritional preparation according to the present invention may be in the form of a ready made liquid or in a powder form or in the form of a troche, a solid composition such as a nutritional bar, a fruit bar, a cookie, a cake, a bread or a muffin.

In another embodiment, a composition according to the present invention is a liquid nutritional preparation in a water-containing liquid, in which the solid ingredients are suspended, dispersed or emulgated in an amount of from 10 to 25 weight percent. When the liquid nutritional preparation is intended for drinking, it will usually comprise a flavoring agent as discussed above. However, the liquid nutritional preparation may also be used for probe administration.

In another embodiment, the present invention relates to the use of a composition according to the present invention as a partial or total diet for an overweight subject, an overweight subject suffering from an arteriosclerotic condition or an overweight subject suffering from a diabetic condition. Obesity is believed to be one of the major causes of diabetes including type 2 diabetes. Overweight subjects, including overweight diabetic subjects, often have increased serum cholesterol levels and increased triglyceride levels and are therefore more likely to develop cardiovascular diseases. However, the present invention is not limited to treating subjects with an increased risk of contracting a cardiovascular disease, i.e. subjects likely to have increased serum levels of cholesterol and/or triglycerides, or to treating obese diabetic subjects with an increased risk of contracting a cardiovascular disease, i.e. obese diabetic subjects likely to have increased serum levels of cholesterol and/or triglycerides. A composition according to the present invention also has substantial serum cholesterol, serum LDL-cholesterol and serum triglyceride lowering effects in subjects having a more normal lipid profile and in diabetic subjects that do not also suffer from overweight. The medical use of a composition according to the present invention is not limited to overweight or obese subjects, including diabetic subjects, but may be used for normal weight subjects having increased serum levels of cholesterol and/or LDL-cholesterol and/or triglycerides or for subjects with a cardiovascular condition such as e.g. arteriosclerosis or a related condition who have normal serum levels of cholesterol and/or LDL-cholesterol and/or triglycerides. Such increased serum levels of cholesterol and/or LDL-cholesterol and/or triglycerides may be caused by intake of a diet rich in fats or it may be genetically related.

For the purpose of the present invention, subjects having an initial total serum cholesterol level of 5.7 mmol/l or below are considered to have a normal or hypocholesterolemic level, whereas subjects having a total serum cholesterol level above 5.7 mmol/l are considered to be hypercholesterolemic. Accordingly, by treating normocholesterolemic subjects, it is possible to prevent the development of cardiovascular diseases arising from serum cholesterol levels below a concentration of 5.7 mmol/l in subjects, including diabetic subjects, particularly sensitive to developing e.g. arteriosclerosis, or prevent further development of cardiovascular diseases in patients, including diabetic patients, with previous cardiovascular events.

By treating hypercholesterolemic subjects, it is possible to prevent the development of cardiovascular diseases arising from serum cholesterol levels above a concentration of 5.7 mmol/l in subjects sensitive to developing e.g. arteriosclerosis under such conditions.

More particularly, subjects having a total serum cholesterol level of from 5.7 mmol/l to 7.9 mmol/l are considered to be mildly hypercholesterolemic. Accordingly, by treating these hypercholesterolemic subjects, it is possible to prevent the development of cardiovascular diseases arising from serum cholesterol levels of from 5.7 to 7.9 mmol/l. Subjects having a total serum cholesterol level of more than 7.9 mmol/l are considered to be severely hypercholesterolemic. Accordingly, by treating these hypercholesterolemic subjects, it is possible to prevent the development of cardiovascular diseases arising from serum cholesterol levels of more than 7.9 mmol/l.

It has also been shown that a composition according to this invention has a potentiating effect to the effect of medications such as e.g. statins. By combining a composition according to the present invention with e.g. statins, such as HMG-CoA-reductase-inhibitors, bile acid resins, fibrates, nicotinic acid derivatives, oat products, such as oat meal, rye products, such as rye meal and various fish oil concentrates with a high content of ω-3-fatty acids, it is possible to achieve a further 5 to 15% reduction in serum levels of total cholesterol and/or LDL-cholesterol and/or triglycerides. The present invention also provides a combination according to the present invention in combination with a statin, preferably an HMG-CoA-reductase-inhibitor, bile acid resins, fibrates, oat products, rye products, nicotinic acid derivatives and various fish oil concentrates with a high content of ω-3-fatty acids.

### EXAMPLE 1

The objective of the present study was to examine if a product comprising isolated soy protein, soybean fibers, and a high, fixed level of isoflavones, is significantly more effective in lowering serum levels of LDL-cholesterol and total cholesterol than placebo. The study was conducted as a randomized, double-blind, placebo controlled trial. Fifty-two patients with a mean baseline cholesterol level of 7.6 mmol/l completed a six-week treatment. Twenty-four consumed a composition according to the invention (Abacor®, Nutri Pharma ASA, Oslo) containing isolated soy protein with high, fixed levels of isoflavones, and soy cotyledon fibers (52 g soy protein, 230 mg soy isoflavones, and 15.5 g soy cotyledon fibers, per day). Twenty-eight consumed a product with the same intakes of protein and fiber, based on casein and cellulose (the placebo). The preparations were given as two daily liquid supplements in addition to the patients regular diets. Both groups were controlled one month after stopping taking the preparations.

The mean reduction of LDL-cholesterol in the Abacor® treated group after six weeks was 13.1%, whereas it was 7.8% (p=0.014) in the placebo treated group. The reduction of total cholesterol was also larger in the active compared to the placebo group (8.4% vs. 5.1%, p=0.049), without correcting for multiple testing. High-density lipoprotein (HDL) cholesterol showed an increase in both the active and placebo groups (6.2% vs. 5.8%). At the one-month follow-up both groups had returned to pre-treatment cholesterol levels.

The results show that intake of a product comprising isolated soy protein with high, fixed levels of isoflavones, and soy cotyledon fibers, significantly reduces serum levels of LDL-cholesterol and total cholesterol, and improves the serum HDL/LDL-cholesterol ratio. The positive results were achieved in this group of patients after six weeks of treatment.

### EXAMPLE 2

The objective of the present study was to examine if a product comprising isolated soy protein, soybean fibers, and a high, fixed level of isoflavones, is significantly more effective in lowering serum levels of LDL-cholesterol, HDL-cholesterol and total cholesterol than placebo. The study was conducted as a randomized, double-blind, placebo controlled trial. 160 patients with plasma levels of LDL-cholesterol ≧4 mM, total cholesterol of 5.8-7.9 mM and triglycerides <4.5 mM completed sixteen weeks treatment. 80 patients divided into two groups of the same size consumed a soy based product (Abacor®, Nutri Pharma ASA, Oslo) containing isolated soy protein with high, fixed levels of isoflavones, and soy cotyledon fibers. Group I (40 patients) received Abacor® I: 18.5 g soy protein (SUPRO® FXP-HO159), 4.63 g soy fibers (FIBRIM® 1020), 3.44 g lecithinated fat reduced cocoa and 1.31 g soy lecithin on a daily basis; Group II (40 patients) received Abacor® II: 31 g soy protein (SUPRO® FXP-HO159), 7.75 g soy fibers (FIBRIM® 1020), 5.77 g lecithinated fat reduced cocoa and 2.2 g soy lecithin on a daily basis. 80 patients divided into two groups of the same size consumed a product with the same intakes of protein and fiber, based on casein, whole milk powder and cellulose (the placebo). The preparations were taken as one daily supplement in addition to the patients regular diets.

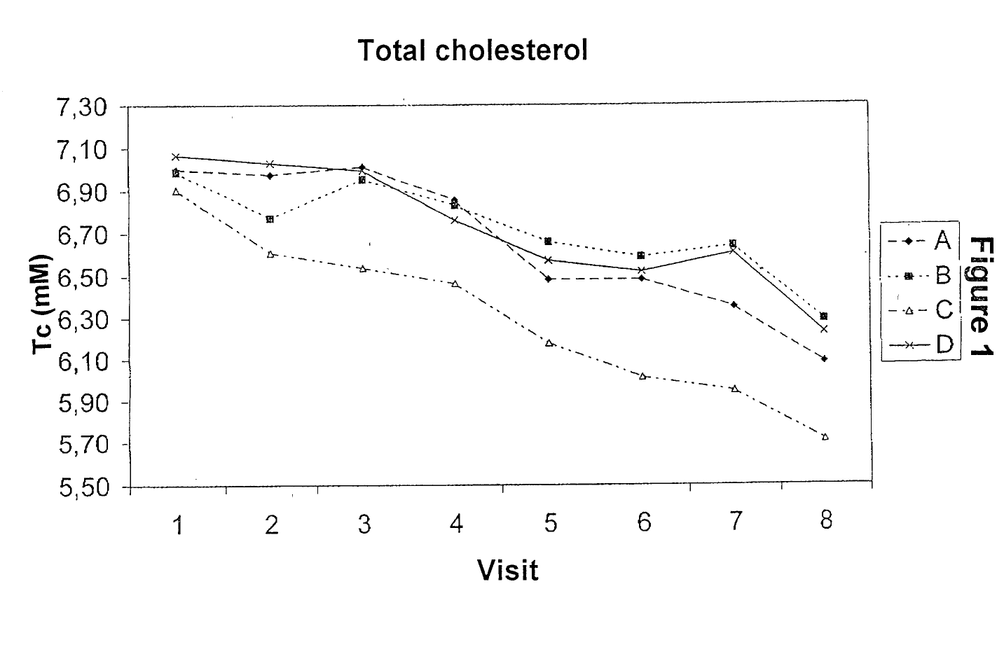

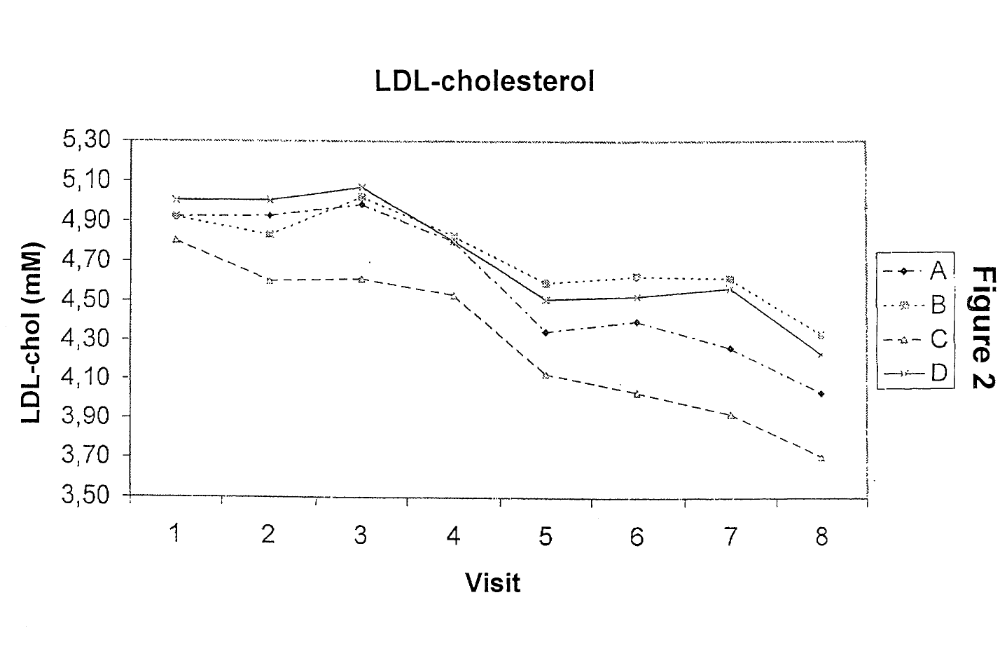

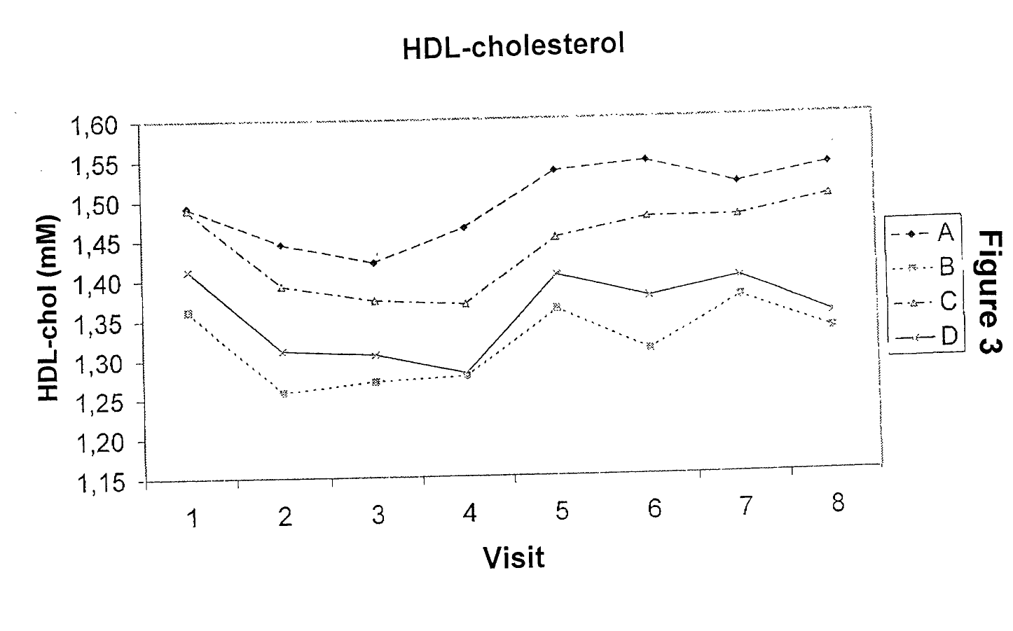

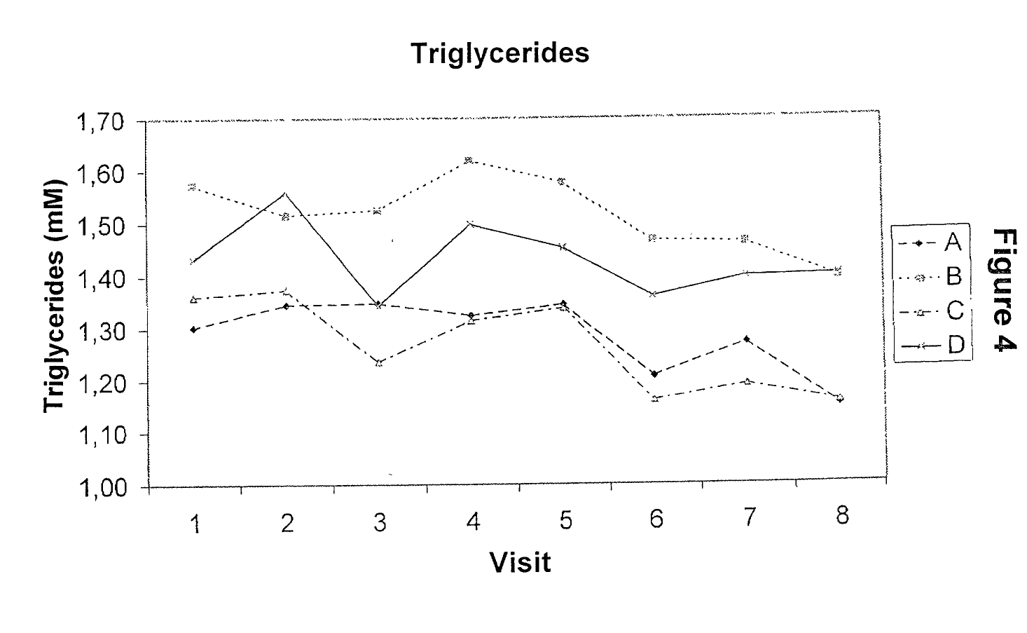

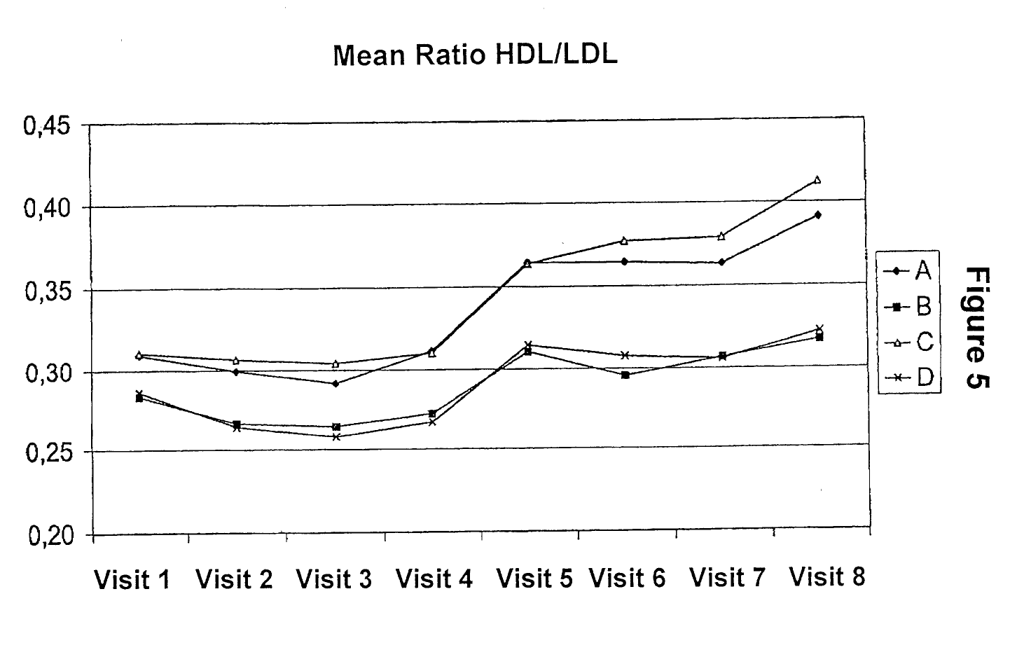

The study began with a standardized dietary programme for on average four months for all study participants (visits 1-4, on average during a course of four months) with extensive education in change of diet and dietary instructions. After this, the study participants received Abacor® or placebo as described above (visits 4-8, one month between each). Serum levels of total cholesterol (Table I and FIG. 1), serum levels of LDL-cholesterol (Table II and FIG. 2), serum levels of HDL-cholesterol (Table III and FIG. 3), and serum levels of triglycerides (Table IV and FIG. 4) were measured at all visits during the entire period. The mean ratio of serum HDL/LDL-cholesterol was also calculated (FIG. 5).

The following designations are used in the following Tables I-IV.

A: Patients in Group I

B: Patients receiving placebo with the same intake of protein and fiber as Group I

C: Patients in Group II

D: Patients receiving placebo with the same intake of protein and fiber as Group II

TABLE I
Total Cholesterol (mM)

| | Visit | Visit | Visit | Visit | Visit | Visit | Visit | Visit | Change |
|---|---|---|---|---|---|---|---|---|---|
| | 1 | 2 | 3 | 4 | 5 | 6 | 7 | 8 | V4-V8 |
| A | 7.00 | 6.98 | 7.01 | 6.86 | 6.48 | 6.48 | 6.35 | 6.08 | 11.3% |
| B | 6.99 | 6.77 | 6.96 | 6.83 | 6.66 | 6.59 | 6.64 | 6.29 | 8.0% |
| C | 6.91 | 6.61 | 6.54 | 6.46 | 6.18 | 6.01 | 5.95 | 5.71 | 11.7% |
| D | 7.07 | 7.03 | 6.99 | 6.76 | 6.58 | 6.52 | 6.61 | 6.23 | 7.9% |

TABLE II
LDL-cholesterol (mM)

| | Visit | Visit | Visit | Visit | Visit | Visit | Visit | Visit | Change |
|---|---|---|---|---|---|---|---|---|---|
| | 1 | 2 | 3 | 4 | 5 | 6 | 7 | 8 | V4-V8 |
| A | 4.92 | 4.93 | 4.99 | 4.80 | 4.35 | 4.40 | 4.26 | 4.03 | 16.0% |
| B | 4.92 | 4.83 | 5.02 | 4.83 | 4.59 | 4.62 | 4.61 | 4.33 | 10.3% |
| C | 4.80 | 4.60 | 4.61 | 4.53 | 4.13 | 4.03 | 3.92 | 3.71 | 18.2% |
| D | 5.01 | 5.01 | 5.07 | 4.80 | 4.51 | 4.52 | 4.56 | 4.23 | 11.9% |

TABLE III
HDL-cholesterol (mM)

| | Visit | Visit | Visit | Visit | Visit | Visit | Visit | Visit | Change |
|---|---|---|---|---|---|---|---|---|---|
| | 1 | 2 | 3 | 4 | 5 | 6 | 7 | 8 | V4-V8 |
| A | 1.49 | 1.44 | 1.42 | 1.46 | 1.53 | 1.54 | 1.52 | 1.54 | −5.0% |
| B | 1.36 | 1.26 | 1.27 | 1.28 | 1.36 | 1.31 | 1.37 | 1.33 | −4.2% |
| C | 1.49 | 1.39 | 1.37 | 1.37 | 1.45 | 1.47 | 1.47 | 1.50 | −9.5% |
| D | 1.41 | 1.31 | 1.31 | 1.28 | 1.40 | 1.37 | 1.40 | 1.35 | −5.4% |

TABLE IV
Triglycerides (mM)

| | Visit | Visit | Visit | Visit | Visit | Visit | Visit | Visit | Change |
|---|---|---|---|---|---|---|---|---|---|
| | 1 | 2 | 3 | 4 | 5 | 6 | 7 | 8 | V4-V8 |
| A | 1.30 | 1.34 | 1.35 | 1.32 | 1.34 | 1.21 | 1.27 | 1.15 | 13.1% |
| B | 1.57 | 1.52 | 1.53 | 1.62 | 1.58 | 1.47 | 1.46 | 1.39 | 13.9% |
| C | 1.36 | 1.37 | 1.24 | 1.31 | 1.34 | 1.16 | 1.19 | 1.16 | 11.9% |
| D | 1.43 | 1.56 | 1.34 | 1.50 | 1.45 | 1.36 | 1.40 | 1.40 | 6.6% |

These results show, that intake of a composition according to the invention in the aforesaid amounts reduces serum levels of total cholesterol with more than 11% for Abacor® I and Abacor® II (Table I and FIG. 1). More remarkably, intake of a composition according to the invention reduces serum levels of LDL-cholesterol with 18.2% for Abacor® II after an initial reduction of about 5% due to change of diet resulting in a total reduction of LDL-cholesterol of about 23% with intake of a composition according to the invention and change of diet (Table II and FIG. 2). This is among the biggest reductions in serum levels of LDL-cholesterol observed with foodstuffs. Surprisingly, intake of Abacor® I (having only 60% of the protein content of Abacor® II) reduces serum levels of LDL-cholesterol with 16.0% after the initial reduction, which is quite close to the result observed with Abacor® II. The results further show, that serum levels of HDL-cholesterol is increased with about 10% for Abacor® II (Table III and FIG. 3), that the serum levels of triglycerides is reduced with more than 10% for both Abacor® I and Abacor® II (Table IV and FIG. 4), and that the serum HDL/LDL-ratio is improved with about 33% for Abacor® II and with about 25% for Abacor® I (FIG. 5).

Surprisingly, the reduction of cholesterol levels caused by the continued use of Abacor® does not stop after approximately one month, as could be expected since the turn-over of cholesterol in the body is 2 to 3 weeks, but the levels of cholesterol are continually reduced over a period of four months (see FIGS. 1 and 2).

### EXAMPLE 3

The study was conducted as a crossover study covering 2×6 weeks. In the first six weeks, one half of the patients received a composition according to the invention (Abalon®, Nutri Pharma ASA, Oslo) containing 50% isolated soy protein, a high, fixed intake of isoflavones (3.7 mg/g protein) and 16.7% soy cotyledon fibers while the other half received a placebo product with the same amounts of protein and fiber. In the second six week period, the patients having received Abalon® in the first period now received the placebo product and vice versa. Every two weeks the patients were examined for some of a wide range of factors, which participate in the metabolic syndrome and type 2 diabetes. The following Tables V-IX summarizes the results of this examination.

TABLE V
Treatment sequence

| | | Active/Placebo | | | | Placebo/Active | | | | |
|---|---|---|---|---|---|---|---|---|---|---|
| Variable | Visit | N | Mean | SD | Wilcoxon | N | Mean | SD | Wilcoxon | Mann-Whitney |
| Total | Visit 1 | 12 | 5.80 | 0.93 | | 8 | 5.85 | 0.90 | | ns |
| chol. | Visit 3 | 12 | 5.15 | 0.87 | | 8 | 5.44 | 0.77 | | ns |
| mmol/l | Diff 1/3 | 12 | 0.65 | 0.44 | 0.0015 | 8 | 0.41 | 0.79 | ns | ns |
| | Visit 4 | 12 | 5.42 | 0.74 | | 8 | 5.51 | 0.72 | | ns |
| | Visit 6 | 12 | 5.46 | 0.98 | | 8 | 5.04 | 0.65 | | ns |
| | Diff 4/6 | 12 | −0.03 | 0.55 | ns | 8 | 0.47 | 0.31 | 0.0156 | 0.0485 |
| HDL- | Visit 1 | 12 | 1.32 | 0.26 | | 8 | 1.20 | 0.19 | | ns |
| chol. | Visit 3 | 12 | 1.42 | 0.43 | | 8 | 1.30 | 0.13 | | ns |
| mmol/l | Diff 1/3 | 12 | −0.10 | 0.23 | ns | 8 | −0.10 | 0.22 | ns | ns |
| | Visit 4 | 12 | 1.33 | 0.34 | | 8 | 1.29 | 0.14 | | ns |
| | Visit 6 | 12 | 1.36 | 0.43 | | 8 | 1.31 | 0.16 | | ns |
| | Diff 4/6 | 12 | −0.02 | 0.18 | ns | 8 | −0.03 | 0.15 | ns | ns |
| LDL-chol. | Visit 1 | 11 | 3.75 | 0.92 | | 7 | 3.90 | 0.86 | | ns |
| mmol/l | Visit 3 | 11 | 3.06 | 0.80 | | 8 | 3.44 | 0.59 | | ns |
| | Diff 1/3 | 11 | 0.69 | 0.36 | 0.0010 | 7 | 0.46 | 0.73 | ns | ns |
| | Visit 4 | 11 | 3.48 | 0.76 | | 8 | 3.46 | 0.54 | | ns |
| | Visit 6 | 11 | 3.25 | 0.83 | | 8 | 2.94 | 0.53 | | ns |
| | Diff 4/6 | 11 | 0.23 | 0.40 | ns | 8 | 0.53 | 0.24 | 0.0078 | ns |
| Triglyce- | Visit 1 | 12 | 1.68 | 1.24 | | 8 | 1.80 | 1.63 | | ns |
| rides | Visit 3 | 12 | 1.57 | 1.02 | | 8 | 1.56 | 0.65 | | ns |
| mmol/l | Diff 1/3 | 12 | 0.12 | 0.44 | ns | 8 | 0.24 | 1.03 | ns | ns |
| | Visit 4 | 12 | 1.63 | 1.45 | | 8 | 1.71 | 1.15 | | ns |
| | Visit 6 | 12 | 1.94 | 1.43 | | 8 | 1.72 | 0.96 | | ns |
| | Diff 4/6 | 12 | −0.31 | 0.44 | 0.0225 | 8 | −0.01 | 0.40 | ns | ns |
| Apolip. | Visit 1 | 12 | 1.07 | 0.21 | | 8 | 1.06 | 0.27 | | ns |
| B 100 | Visit 3 | 12 | 0.82 | 0.20 | | 8 | 0.95 | 0.29 | | ns |
| mg/dl | Diff 1/3 | 12 | 0.26 | 0.12 | 0.0005 | 8 | 0.11 | 0.25 | ns | ns |
| | Visit 4 | 12 | 0.87 | 0.22 | | 8 | 1.11 | 0.21 | | 0.0265 |
| | Visit 6 | 12 | 1.01 | 0.22 | | 8 | 0.93 | 0.17 | | ns |
| | Diff 4/6 | 12 | −0.14 | 0.23 | ns | 8 | 0.19 | 0.14 | 0.0156 | 0.0022 |
| Homo- | Visit 1 | 12 | 11.38 | 4.67 | | 8 | 10.68 | 2.40 | | ns |
| cystein | Visit 3 | 12 | 11.48 | 4.74 | | 8 | 11.80 | 2.48 | | ns |
| μmol/l | Diff 1/3 | 12 | −0.09 | 1.22 | ns | 8 | −1.13 | 0.84 | 0.0234 | ns |
| | Visit 4 | 12 | 10.52 | 2.80 | | 8 | 10.88 | 2.61 | | ns |
| | Visit 6 | 12 | 13.27 | 5.78 | | 8 | 11.70 | 2.75 | | ns |
| | Diff 4/6 | 12 | −2.74 | 3.17 | 0.0005 | 8 | −0.83 | 0.71 | 0.0313 | 0.0163 |

In this table, Active/Placebo refers to patients having received Abalon ® during the first six weeks (visits 1-3) and placebo the last six weeks while Placebo/Active refers to the opposite situation; ns is not significant and Wilcoxon and Mann-Whitney are the used statistical methods.

TABLE VI

| | | Active period | | | | Placebo period | | | | Difference | | | |
|---|---|---|---|---|---|---|---|---|---|---|---|---|---|
| Vari. | Visit | N | Mean | SD | Wilcoxon | N | Mean | SD | Wilcoxon | N | Mean | SD | Wilcoxon |
| Total | Visit 1 | 20 | 5.68 | 0.84 | | 20 | 5.59 | 0.81 | | 20 | 0.09 | 0.66 | ns |
| chol. | Visit 3 | 20 | 5.11 | 0.78 | | 20 | 5.45 | 0.88 | | 20 | −0.34 | 0.50 | 0.0041 |
| | Diff 1/3 | 20 | 0.58 | 0.39 | 0.0001 | 20 | 0.14 | 0.68 | ns | 20 | 0.43 | 0.90 | ns |
| HDL- | Visit 1 | 20 | 1.31 | 0.22 | | 20 | 1.28 | 0.29 | | 20 | 0.03 | 0.19 | ns |
| chol. | Visit 3 | 20 | 1.38 | 0.35 | | 20 | 1.33 | 0.34 | | 20 | 0.04 | 0.14 | ns |
| | Diff 1/3 | 20 | −0.07 | 0.20 | ns | 20 | −0.05 | 0.20 | ns | 20 | −0.02 | 0.24 | ns |
| LDL- | Visit 1 | 19 | 3.63 | 0.78 | | 18 | 3.64 | 0.80 | | 18 | −0.00 | 0.66 | ns |
| chol. | Visit 3 | 19 | 3.01 | 0.68 | | 19 | 3.33 | 0.72 | | 19 | −0.32 | 0.40 | 0.0044 |
| | Diff 1/3 | 19 | 0.62 | 0.32 | 0.0001 | 18 | 0.32 | 0.54 | 0.0220 | 18 | 0.32 | 0.67 | ns |
| TG | Visit 1 | 20 | 1.70 | 1.17 | | 20 | 1.70 | 1.49 | | 20 | −0.01 | 0.58 | ns |
| | Visit 3 | 20 | 1.63 | 0.97 | | 20 | 1.79 | 1.17 | | 20 | −0.16 | 0.57 | ns |
| | Diff 1/3 | 20 | 0.07 | 0.42 | ns | 20 | −0.09 | 0.76 | ns | 20 | 0.16 | 0.78 | ns |
| Apo B | Visit 1 | 20 | 1.09 | 0.21 | | 20 | 0.95 | 0.26 | | 20 | 0.15 | 0.29 | ns |
| | Visit 3 | 20 | 0.86 | 0.19 | | 20 | 0.98 | 0.25 | | 20 | −0.12 | 0.22 | 0.0249 |
| | Diff 1/3 | 20 | 0.23 | 0.13 | 0.0001 | 20 | −0.04 | 0.26 | ns | 20 | 0.27 | 0.35 | 0.0026 |
| Homo- | Visit 1 | 20 | 11.18 | 3.90 | | 20 | 10.58 | 2.58 | | 20 | 0.59 | 1.90 | ns |
| cys | Visit 3 | 20 | 11.57 | 3.97 | | 20 | 12.68 | 4.71 | | 20 | −1.11 | 1.58 | 0.0040 |
| | Diff 1/3 | 20 | −0.38 | 1.09 | ns | 20 | −2.10 | 2.60 | 0.0001 | 20 | 1.71 | 2.98 | 0.0057 |

In this table TG = triglycerides, Apo B = apolipoprotein B, Homo-cys = homocysteine, ns = not significant, Wilcoxon is the used statistical method and all units like in Table I

TABLE VII

| | % change | | |
|---|---|---|---|
| Variable | Active | Placebo | Difference |
| Total cholesterol | −10.1 | −2.5 | 7.6 |
| HDL-cholesterol | 5.3 | 3.9 | 1.4 |
| Triglycerides | −4.1 | 5.3 | 9.4 |
| Apolipo. B 100 | −21.1 | 4.2 | 25.3 |
| Homocystein | 3.4 | 19.8 | 16.4 |

TABLE VIII

| | | Treatment | | | | | | | | |
|---|---|---|---|---|---|---|---|---|---|---|
| | | Active | | | | Placebo | | | | |
| Variable | Visit | N | Mean | SD | Wilcoxon | N | Mean | SD | Wilcoxon | Mann-Whitney |
| Glucose | Visit 1 | 12 | 2736 | 872 | | 8 | 2360 | 772 | | 0.1325 |
| mmol/l | Visit 4 | 12 | 2903 | 1159 | | 8 | 2478 | 1021 | | 0.3749 |
| | Diff 1/4 | 12 | 167 | 472 | 0.4697 | 8 | 117 | 294 | 0.6406 | 0.9079 |
| Insulin | Visit 1 | 12 | 44436 | 28492 | | 8 | 47125 | 19356 | | 0.5120 |
| IU/ml | Visit 4 | 12 | 45057 | 29561 | | 8 | 42405 | 19488 | | 0.9692 |
| | Diff 1/4 | 12 | 621 | 7682 | 0.8501 | 8 | −4720 | 6019 | 0.0781 | 0.1325 |

IU = international units, Wilcoxon and Mann-Whitney are the used statistical methods

TABLE IX

| | | Treatment sequence | | | | | | | | |
|---|---|---|---|---|---|---|---|---|---|---|
| | | Active/Placebo | | | | Placebo/Active | | | | |
| Variable | Visit | N | Mean | SD | Wilcoxon | N | Mean | SD | Wilcoxon | Mann-Whitney |
| Glucose | 3-1 | 12 | 204 | 379 | 0.1099 | 8 | 189 | 223 | 0.0078 | 0.9692 |
| mmol/l | 6-4 | 12 | 284 | 441 | 0.0269 | 8 | −16 | 147 | 0.8437 | 0.0409 |
| Insulin | 3-1 | 12 | 26323 | 40064 | 0.0005 | 8 | 27078 | 28502 | 0.0078 | 0.7285 |
| IU/ml | 6-4 | 12 | 28377 | 30798 | 0.0010 | 8 | 26267 | 22637 | 0.0078 | 0.9079 |

IU = international units, Wilcoxon and Mann-Whitney are the used statistical methods

In this study it is shown that Abalon® in the given dosage and for the given time reduces the amount of total cholesterol, reduces the amount of LDL-cholesterol, reduces the amount of Apolipoprotein B 100, and results in a different level of homocystein than placebo. A significant difference between the Abalon® group and the placebo group with regards to total cholesterol was expected, but the low number of patients and the design of the study have probably blurred the results. The same can be said for the levels of glucose and insulin, where a longer period of study probably would have revealed a larger difference between Abalon® and placebo. Other unpublished studies regarding the cholesterol lowering effect of a composition according to the invention shows, that the effect gets more and more pronounced with time (up to four months).

### EXAMPLE4

A 52 years old man, who had suffered from chronic asthma for many years, was treated with two daily supplements of a composition according to the invention comprising isolated soy protein, isoflavones and soy cotyledon fibers. The daily dose of isolated soy protein was 25 g and the daily dose of soy cotyledon fibers was 8 g. This treatment led to a reduced intake of medicine and a substantial clinical improvement with fewer and less severe attacks of asthma and improved lung capacity.

### EXAMPLE 5

A 19 years old man, who suffered from chronic asthma, received the same dosage of the same composition as the subject in Example 1. This led to reduced intake of medicine and substantially improved capacity to perform physical activity. This subject was for the first time in years able to run and play football, which in the past immediately and inevitably provoked attacks of asthma.

1 (amended). A composition comprising
(a) a soy protein source, selected from isolated soy protein, soy protein concentrate, or soy flour, said soy protein source providing an amount of soy protein, which is at least 45 weight percent of the total protein content of the composition, said total protein content providing at least 15 percent of the total energy content of the composition,
(b) at least one phytoestrogen compound, said phytoestrogen compounds being present in a total amount of more than 0.16 weight percent of the soy protein content of the composition, and
(c) dietary fibers in an amount of more than 6 weight percent of the total weight of the nutritional composition on a dry basis.

2. A composition according to claim 1, wherein the soy protein source is isolated soy protein and the amount of isolated soy protein is at least 50 weight percent of the total protein content.

3. A composition according to claim 2, wherein the amount of isolated soy protein is at least 75 weight percent of the total protein content.

4. A composition according to claim 3, wherein the amount of isolated soy protein is at least 90 weight percent of the total protein content.

5. A composition according to claim 4, wherein substantially all of the protein is isolated soy protein.

6. A composition according to claim 1, wherein the soy protein source is soy protein concentrate or soy flour and the amount of soy protein is at least 50 weight percent of the total protein content.

7. A composition according to claim 6, wherein the amount of soy protein is at least 75 weight percent of the total protein content.

8. A composition according to claim 7, wherein the amount of soy protein is at least 90 weight percent of the total protein content.

9. A composition according to claim 8, wherein substantially all of the protein is soy protein.

10. A composition according to claim 1, wherein the dietary fibers are soybean fibers.

11. A composition according to claim 10, wherein the soybean fibers are soy cotyledon fibers.

12. A composition according to claim 1 wherein the phytoestrogen compound is present in an amount of at least about 0.20 weight percent of the soy protein content of the composition.

13. A composition according to claim 12 wherein the phytoestrogen compounds are present in a total amount of at least about 0.30 weight percent of the soy protein content of the composition.

14. A composition according to claim 13 wherein the phytoestrogen compounds are present in a total amount of at least about 0.33 weight percent of the soy protein content of the composition.

15. A composition according to claim 14 wherein the phytoestrogen compounds are present in a total amount of at least 0.45 weight percent of the soy protein content of the composition.

16. A composition according to claim 15 wherein the phytoestrogen compounds are present in a total amount of at least about 0.75 weight percent of the soy protein content of the composition.

17. A composition according to claim 16 wherein the phytoestrogen compounds are present in a total amount of at least about 1.0 weight percent of the soy protein content of the composition.

18. A composition according to claim 1 wherein at least one of the phytoestrogen compounds is an isoflavone.

19. A composition according to claim 18 wherein at least one of the phytoestrogen compounds is an isoflavone selected from the group consisting of genistein, daidzein, glycitein and equol.

20. A composition according to claim 19 where at least one of the phytoestrogen compounds is genistein or daidzein.

21. A composition according to claim 20 wherein at least one of the phytoestrogen compounds is genistein.

22. A composition according to claim 18 wherein some or all of the isoflavones are present in the aglycone form.

23. A composition according to claim 1 wherein the dietary fibers are present in an amount of at least 7 weight percent of the composition.

24. A composition according to claim 1 wherein the weight ratio of soy protein of dietary fibers is at least about 1.0.

25. A composition according to claim 24 wherein the weight ratio of soy protein to dietary fibers is at least about 1.5.

26. A composition according to claim 25 wherein the weight ratio of soy protein to dietary fibers is at least about 2.0.

27. A composition according to claim 26 wherein the weight ratio of soy protein to dietary fibers is at least about 2.5.

28. A composition according to claim 27 wherein the weight ratio of soy protein to dietary fibers is at least about 3.0.

29. A composition according to claim 28 wherein the weight ratio of soy protein to dietary fibers is at least about 4.0.

30. A composition according to claim 29 wherein the weight ratio of soy protein to dietary fibers is at least about 5.0.

31. A composition according to claim 1, further comprising one or more statins, bile acid resins, fibrates, nicotinic acid derivatives, oat products, rye products or fish oil concentrates with a high content of ω-3-fatty acids, or any combination thereof.

32. A composition according to claim 31 wherein the statins are HMG-CoA-reductase-inhibitors.

33. A composition according to claim 1 further comprising an additional protein source and/or an additional carbohydrate source and/or an additional fat source.

34. A composition according to claim 1 in the form of a micronutrient.

35. A composition according to claim 34 additionally comprising a DNA topoisomerase inhibitor, a ribosome kinase inhibitor, and/or a growth control factor.

36. A composition according to claim 35 wherein the growth control factor is a growth control factor controllable by a tyrosine kinase activity.

37. A composition according to claim 34 additionally comprising ormeloxifene and/or levormeloxifene.

38. A composition according to claim 1, further comprising a functional food ingredient comprising a sterol.

39. A composition according to claim 38 wherein the functional food ingredient comprising a sterol is selected from the group consisting of food ingredients comprising a stanol ester, a tocotrienol, a mevinolin, and a phytosterol compound, or a combination thereof.

40. A composition according to claim 1, suitable for use as a food or as an ingredient of a food.

41. A composition according to claim 40, where the food is selected from the group consisting of dairy products, juice, ready make liquids for drinking, a spreadable product, a cereal product, nutritional bars, biscuits, bread, soups, meat products, meat substitute products, and a vegetable product.

42. A composition according to claim 1 which is classified by the U.S. Food and Drug Administration as a food for special dietary use.

43. A method of preventing, treating, prophylactically treating and/or alleviating by therapy a cardiovascular disease in a human or animal body, said method comprising administration to said human or animal body of a composition according to claim 1 in an amount effective in lowering serum levels of total cholesterol and/or LDL-cholesterol and/or triglycerides and/or homocystein and/or increasing the serum HGL/LDL-cholesterol ratio and/or serum HDL-cholesterol levels and/or reducing the influx of cholesterol and/or triglycerides into the arterial wall and/or reducing the amount of oxidized LDL-cholesterol present in the arterial wall and/or preventing, reducing or eliminating fatty streak formation and/or preventing, reducing or eliminating fibrous plaque formation and/or preventing, reducing or eliminating complicated lesion formation and/or reducing or eliminating the risk of a subject contracting angina pectoris and/or reducing or eliminating the risk of subject contracting a myocardial infarction and/or alleviating the clinical condition of patients contracting a myocardial infection.

44. A method according to claim 43 wherein the cardiovascular disease is an arteriosclerotic condition of the human or animal body.

45. A method according to claim 43 wherein the cardiovascular disease is selected from the group consisting of hypercholesterolemia, hypertriglyceridemia, other hyperlipidemias, arteriosclerosis, atherosclerosis, arteriolosclerosis, coronary heart disease, angina pectoris, thrombosis, myocardial infarction, and hypertension.

46. A method according to claim 45 wherein the cardiovascular disease is arteriosclerosis.

47. A method according to claim 45 wherein the cardiovascular disease is atherosclerosis.

48. Method of preventing and/or treating by therapy type 2 diabetes in a human or animal body, said method comprising administration to said human or animal body of a composition according to claim 1 in an amount effective in lowering serum levels of glucose and/or total cholesterol and/or LDL-cholesterol and/or triglycerides and/or homocystein and/or reducing the influx of cholesterol and/or triglycerides into the arterial wall and/or reducing the amount of oxidized LDL-cholesterol present in the arterial wall and/or improving glucose tolerance and/or increasing insulin sensitivity and/or alleviating impaired glucose tolerance and/or improving insulin secretion and/or reducing or eliminating fatty streak formation and/or preventing, reducing or eliminating fibrous plaque formation and/or preventing, reducing or eliminating complicated lesion formation and/or preventing, reducing or eliminating complicated lesion formation and/or preventing, reducing or eliminating the risk of a diabetic subject contracting angina pectoris and/or preventing, reducing or eliminating the risk of a diabetic subject contracting a myocardial infarction and/or preventing, treating, prophylactically treating, alleviating and/or eliminating hypertension and/or hyperglycemia and/or hyperinsulinemia and/or hypercholesterolemia and/or hypertriglyceridemia and/or arteriosclerosis and/or atherosclerosis and/or arteriolosclerosis in a diabetic subject.

49. Method of preventing and/or treating by therapy the metabolic syndrome in a human or animal body, said method comprising administration to said human or animal body of a composition according to claim 1 in an amount effective in lowering serum levels of glucose and/or total cholesterol and/or LDL-cholesterol and/or triglycerides and/or homocystein and/or reducing the influx of cholesterol and/or triglycerides into the arterial wall and/or reducing the amount of oxidized LDL-cholesterol present in the arterial wall and/or improving glucose tolerance and/or increasing insulin sensitivity and/or alleviating impaired glucose tolerance and/or improving insulin secretion and/or reducing or eliminating fatty streak formation and/or preventing, reducing or eliminating fibrous plaque formation and/or preventing, reducing or eliminating complicated lesion formation and/or preventing, reducing or eliminating the risk of a subject contracting angina pectoris and/or preventing, reducing or eliminating the risk of a subject contracting a myocardial infarction.

50. A composition according to claim 33 wherein the composition comprises an additional fat source.

51. The composition of claim 50 where the additional fat source comprises a soy lecithin.

52. A composition according to claim 50 wherein the additional fat source comprises a polyunsaturated fatty acid or a monounsaturated fatty acid.

53. A composition according to claim 1 wherein the phytoestrogen compounds are present in a total amount of at least about 0.18 weight percent of the soy protein content of the composition.

54. A composition according to claim 1 wherein the phytoestrogen compounds are present in a total amount of at least about 0.55 weight percent of the soy protein content of the composition.

55. A composition comprising
(a) a soy protein source, selected from isolated soy protein, soy protein concentrate, or soy flour, said soy protein source providing an amount of soy protein, which is at least 45 weight percent of the total protein content of the composition, said total protein content providing at least 15 percent of the total energy content of the composition,
(b) at least one phytoestrogen compound, said phytoestrogen compounds being present in a total amount of more than 0.55 weight percent of the soy protein content of the composition, and
(c) dietary fibers in an amount of more than 4 weight percent of the total weight of the nutritional composition on a dry basis.
